(19) United States
(12) Patent Application Publication
(10) Pub. No.: US 2023/0355389 A1
(43) Pub. Date: Nov. 9, 2023

(54) EXPANDABLE SHEATH

(71) Applicant: EDWARDS LIFESCIENCES CORPORATION, Irvine, CA (US)

(72) Inventors: Sovanpheap Mak, Santa Ana, CA (US); Ilan Tamir, Irvine, CA (US); Maxwell Harrison Fine, Costa Mesa, CA (US); Nasser William Saleh, Stockton, CA (US); Sonny Tran, Westminster, CA (US)

(21) Appl. No.: 18/354,566

(22) Filed: Jul. 18, 2023

Related U.S. Application Data
(63) Continuation of application No. PCT/US2022/012785, filed on Jan. 18, 2022.
(60) Provisional application No. 63/138,923, filed on Jan. 19, 2021.

(51) Int. Cl. A61F 2/24

(52) U.S. Cl. CPC A61F 2/2436; A61F 2002/9623

(57) ABSTRACT

An expandable sheath including an elongated inner member defining a central lumen extending therethrough. The inner member includes a first circumferential portion including a first and second longitudinal edges, and a second circumferential portion extending between the first and second longitudinal edges. The wall thickness of the first circumferential portion is greater than the wall thickness of the second circumferential portion. The elongated inner member is configured to transition from an unexpanded configuration to an expanded configuration in response to a radially outward force on an inner surface of the central lumen. In the unexpanded configuration, the elongated inner member is creased proximate the first and second longitudinal edges into a folded configuration such that the second circumferential portion is positioned at least partially radially inward of the first circumferential portion.

### RELATED APPLICATIONS

This application is a continuation of PCT patent application no. PCT/US2022/012785 filed on Jan. 18, 2022, which claims the benefit of U.S. Provisional Application 63/138,923, filed Jan. 19, 2021, each of which is incorporated herein by reference in its entirety.

### FIELD

The present application is directed to a sheath for use with catheter-based technologies for repairing and/or replacing heart valves, as well as for delivering an implant, such as a prosthetic valve to a heart via a subject's vasculature.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques. Percutaneous interventional medical procedures utilize the large blood vessels of the body reach target destinations rather than surgically opening target site. There are many types of diseases states that can be treated via interventional methods including coronary blockages, valve replacements (TAVR) and brain aneurysms. These techniques involve using wires, catheters, balloons, electrodes and other thin devices to travel down the length of the blood vessels from the access site to the target site. The devices have a proximal end which the clinician controls outside of the body and a distal end inside the body which is responsible for treating the disease state. Percutaneous interventional procedures offer several advantages over open surgical techniques. First, they require smaller incision sites which reduces scarring and bleeding as well as infection risk. Procedures are also less traumatic to the tissue, so recovery times are reduced. Finally, interventional techniques can usually be performed much faster, and with fewer clinicians participating in the procedure, so overall costs are lowered. In some cases, the need for anesthesia is also eliminated, further speeding up the recovery process and reducing risk.

A single procedure typically uses several different guidewires, catheters, and balloons to achieve the desired effect. One at a time, each tool is inserted and then removed from the access site sequentially. For example, a guidewire is used to track to the correct location within the body. Next a balloon may be used to dilate a section of narrowed blood vessel. Last, an implant may be delivered to the target site. Because catheters are frequently inserted and removed, introducer sheaths are used to protect the local anatomy and simplify the procedure.

An introducer sheath can be used to safely introduce a delivery apparatus into a subject's vasculature (e.g., the femoral artery). Introducer sheaths are conduits that seal onto the access site blood vessel to reduce bleeding and trauma to the vessel caused by catheters with rough edges. An introducer sheath generally has an elongated sleeve that is inserted into the vasculature and a housing that contains one or more sealing valves that allow a delivery apparatus to be placed in fluid communication with the vasculature with minimal blood loss. Once the introducer sheath is positioned within the vasculature, the shaft of the delivery apparatus is advanced through the sheath and into the vasculature, carrying the prosthetic device. Expandable introducer sheaths, formed of highly elastomeric materials, allow for the dilating of the vessel to be performed by the passing prosthetic device. Expandable introducer sheaths are disclosed in U.S. Pat. No. 8,790,387, entitled “Expandable Sheath for Introducing an Endovascular Delivery Device into a Body,” U.S. Pat. No. 10,639,152, entitled “Expandable Sheath and Methods of Using the Same,” U.S. application Ser. No. 14/880,109, entitled “Expandable Sheath,” U.S. application Ser. No. 16/407,057, entitled “Expandable Sheath with Elastomeric Cross Sectional Portions,” U.S. Pat. No. 10,327,896, entitled “Expandable Sheath with Elastomeric Cross Sectional Portions,” U.S. application Ser. No. 15/997,587, entitled “Expandable Sheath for Introducing an Endovascular Delivery Device into a Body,” U.S. application Ser. No. 16/378,417, entitled “Expandable Sheath,” U.S. Provisional Patent Application No. 62/912,569, entitled “Expandable Sheath,” and U.S. Provisional Patent Application No. 63/091,722, entitled “Radiopaque Foil Encapsulated within an expandable Sheath” the disclosures of which are herein incorporated by reference.

Conventional methods of accessing a vessel, such as a femoral artery, prior to introducing the delivery system include dilating the vessel using multiple dilators or sheaths that progressively increase in diameter. Typically, the introducer is inserted into the sheath during preparation and both are then inserted into the vessel. Some procedures, such as a transseptal approach for mitral valve replacement/repair, require prolonged dilation of incisions in heart tissue and a curving/bending of the sheath to access the treatment site, prolonging procedure time and recovering and increasing risk of trauma to vessels and heart tissue.

The use of radially expanding introducer sheaths reduce the overall profile of the sheath to reduce risk of damage to the vessel. Such sheaths tend to have complex mechanisms, such as ratcheting mechanisms that maintain the shaft or sheath in an expanded configuration once a device with a larger diameter than the sheath's original diameter is introduced. However, delivery and/or removal of prosthetic devices and other material to or from a patient still poses a risk to the patient. Furthermore, accessing the vessel remains a challenge due to the relatively large profile of the delivery system that can cause longitudinal and radial tearing of the vessel during insertion. The delivery system can additionally dislodge calcified plaque within the vessels, posing an additional risk of clots caused by the dislodged plaque. The addition of radially expanding properties can also hinder a practitioner's ability to push the sheath without it bending or kinking. Thus, there remains a need for further improvements in introducer sheaths for endovascular systems used for implanting heart valves and other prosthetic devices.

### SUMMARY

Disclosed herein are expandable introducer sheaths and methods of making and using the same. The expandable introducer sheaths disclosed herein are used to deliver a prosthetic device through a subject's vasculature to a procedure site within the body. The sheath is constructed to be highly expandable and collapsible in the circumferential direction, while also minimizing the wall thickness of the sheath to minimize the profile of the delivery system.

Introducer sheaths and delivery devices may be utilized in a variety of subjects and procedures. Subjects include (but are not limited to) medical patients, veterinary patients, animal models, cadavers, and simulators of the cardiac and vasculature system (e.g., anthropomorphic phantoms and explant tissue). Procedures include (but are not limited to) medical and training procedures.

Some embodiments include an expandable sheath including an elongated inner member defining a central lumen, a first circumferential portion including a first and second longitudinal edges, and a second circumferential portion extending between the first and second longitudinal edges, the first circumferential portion having a wall thickness greater than a wall thickness of the second circumferential portion; wherein the elongated inner member is configured to transition from an unexpanded configuration to an expanded configuration in response to a radially outward force on an inner surface of the central lumen; wherein, in the unexpanded configuration, the elongated inner member is creased proximate the first and second longitudinal edges into a folded configuration such that the second circumferential portion is positioned at least partially radially inward of the first circumferential portion.

Some embodiments include a method of making an expandable sheath, the method comprises forming an inner member having portions of varying wall thickness including a first circumferential portion having a first and second longitudinal edges, and a second circumferential portion extending between the first and second longitudinal edges, the first circumferential portion having a wall thickness greater than a wall thickness of the second circumferential portion; and disposing an outer elastomeric member over the inner member to form the sheath; wherein the sheath is configured to transition from an unexpanded configuration to an expanded configuration in response to a radially outward force on an inner surface of a central lumen of the inner member, wherein the outer elastomeric member urges the inner member towards the unexpanded configuration, wherein, in the unexpanded configuration the elongated inner member is creased proximate the first and second longitudinal edges into a folded configuration such that the second circumferential portion is positioned at least partially radially inward of the first circumferential portion.

Some embodiments include a method of delivering a prosthetic device to a procedure site, the method comprises: positioning an expandable sheath within the vascular system of a patient; introducing a prosthetic device into a lumen of the expandable sheath; advancing the prosthetic device through the lumen of the expandable sheath such that the prosthetic device exerts a radially outward force on an inner surface of an inner member of the expandable sheath and locally unfolds the inner member into an expanded configuration; advancing the prosthetic device further through the lumen and through a distal tip portion of the expandable sheath locally enlarging a lumen of the distal tip portion in response to radial pressure exerted by passage of the prosthetic device; at least partially collapsing the inner member and the distal tip portion after the prosthetic device has passed in response to a radially inward force provided by an outer elastomeric member of the sheath; and delivering the prosthetic device to the procedure site.

### DESCRIPTION OF DRAWINGS

FIGS. 1A-1C show side elevation views of an expandable introducer sheath (FIG. 1C) and a delivery apparatus for deployment through the sheath (FIGS. 1A-1B).

FIG. 2 shows a perspective view of an expandable introducer sheath.

FIG. 3 shows a cross-sectional view of the expandable introducer sheath of FIG. 2 in an unexpanded configuration.

FIG. 4 shows a cross-sectional view of the expandable introducer sheath of FIG. 2 in an expanded configuration.

FIG. 5 shows an enlarged a partial cross-section view of the expandable introducer sheath of FIG. 4.

FIG. 6 shows an enlarged a partial cross-section view of the expandable introducer sheath of FIG. 4 according to another embodiment.

FIG. 7 shows an enlarged a partial cross-section view of the expandable introducer sheath of FIG. 4.

FIG. 8 shows an enlarged a partial cross-section view of the expandable introducer sheath of FIG. 4 according to another embodiment.

FIG. 9 shows an enlarged a partial cross-section view of the expandable introducer sheath of FIG. 4 according to another embodiment.

FIG. 10 shows the enlarged a partial cross-section view of FIG. 7 with a lubricant applied in a regular pattern around the circumference of the sheath.

FIGS. 11A-11D show partial elevation views of various exemplary embodiments of a braid structure with various PIC according to the present disclosure.

FIGS. 12A-12B show a block diagram of one embodiment of a method of making a sheath according to the present disclosure.

FIG. 13 shows an end view of an inner member during a manufacturing process.

FIG. 14 shows the top view of the inner member of FIG. 11.

FIG. 15 shows a perspective view of the inner member of FIG. 11.

FIG. 16 shows an end view of an inner member and liner during a manufacturing process.

FIG. 17 shows a top view of the inner member and liner of FIG. 16.

FIG. 18 shows a perspective view of the inner member and liner of FIG. 16.

FIG. 19 shows an end view of an example mandrel.

FIG. 20 shows an end view of the mandrel with the inner member and liner mounted thereon.

### DETAILED DESCRIPTION

The following description of certain examples of the inventive concepts should not be used to limit the scope of the claims. Other examples, features, aspects, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the embodiments of this disclosure are described herein. The described methods, systems, and apparatus should not be construed as limiting in any way. Instead, the present disclosure is directed toward all novel and nonobvious features and aspects of the various disclosed embodiments, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect, embodiment or example of the invention are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The terms “proximal” and “distal” as used herein refer to regions of a sheath, catheter, or delivery assembly. “Proximal” means that region closest to handle of the device, while “distal” means that region farthest away from the handle of the device.

“Axially” or “axial” as used herein refers to a direction along the longitudinal axis of the sheath.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

The term “tube” or “tubular” as used herein is not meant to limit shapes to circular cross-sections. Instead, tube or tubular can refer to any elongate structure with a closed-cross section and lumen extending axially therethrough. A tube may also have some selectively located slits or openings therein—although it still will provide enough of a closed structure to contain other components within its lumen(s).

The expandable introducer sheath disclosed herein is used to deliver a prosthetic device through a subject's vasculature to a procedure site within the body. The sheath is constructed to be highly expandable and collapsible in the circumferential direction, while also minimizing the wall thickness of the sheath to minimize the profile of the delivery system. These expandable introducer sheaths that are particularly suitable for use in the delivery of implants in the form of implantable heart valves, such as balloon-expandable implantable heart valves. Balloon-expandable implantable heart valves are well-known and will not be described in detail here. An example of such an implantable heart valve is described in U.S. Pat. No. 5,411,552, entitled “Valve Prosthesis for Implantation in the Body and a Catheter for Implanting Such Valve Prosthesis,” and also in U.S. Patent Application Publication No. 2012/0123529, entitled “Prosthetic Heart Valve,” both of which are hereby incorporated by reference. The expandable introducer sheaths disclosed herein can also be used with the delivery systems for other types of implantable devices, such as self-expanding implantable heart valves, stents or filters. The term “implantable” as used herein is broadly defined to mean anything—prosthetic or not—that is delivered to a site within a body. A diagnostic device, for example, can be implantable.

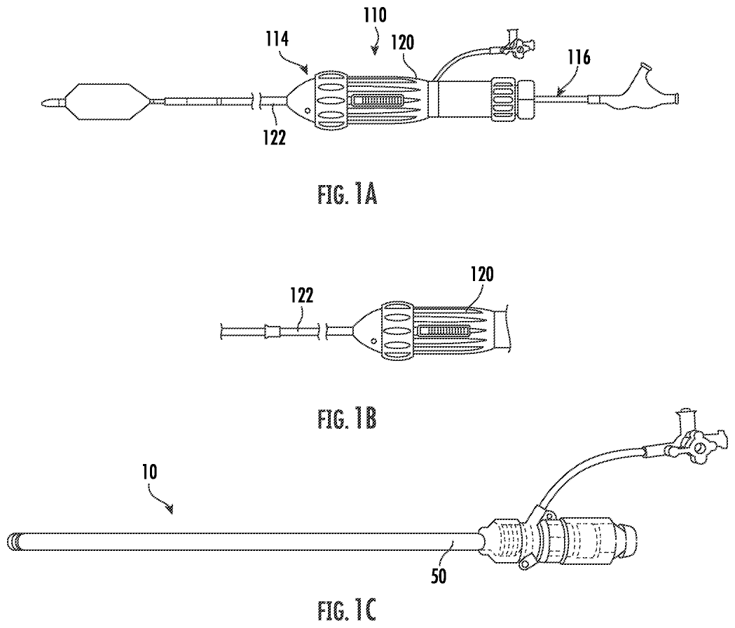

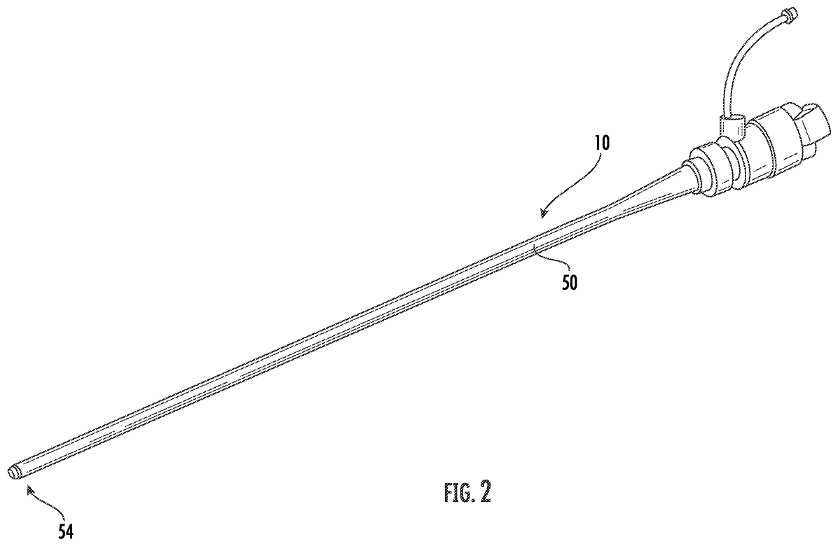

FIGS. 1A-1C illustrate an expandable introducer sheath 10 according to the present disclosure and a representative delivery apparatus 110 for delivering a prosthetic implant, such as a prosthetic heart valve, to a subject. It should be understood that the delivery apparatus 110 described herein is exemplary only, and that other similar delivery systems can of course be used with the expandable sheath 10. The delivery apparatus 110 illustrated herein generally includes a steerable guide catheter 114 and a balloon catheter 116 extending through the guide catheter 114.

The guide catheter 114 and the balloon catheter 116 illustrated in FIGS. 1A-1B are adapted to slide longitudinally relative to each other to facilitate delivery and positioning of prosthetic heart valve at an implantation site in a subject, as described in detail below. The guide catheter 114 includes a handle portion 120 and an elongated guide tube, or shaft, 122 extending from handle portion 120 (FIG. 1B). Additional examples of introducer devices and expandable sheaths can be found in U.S. Patent Publication No. 2019/0307589, entitled “Expandable Sheath,” and U.S. Provisional Patent Application No. 62/912,569, entitled “Expandable Sheath,” which are incorporated by reference in their entireties.

FIG. 1C illustrates an expandable sheath 10 that is used to introduce the delivery apparatus 110 and the prosthetic device into the subject. The expandable sheath 10 has generally tubular configuration defining a central lumen to guide passage of the delivery system for the prosthetic heart valve. At a proximal end, the expandable sheath 10 includes a hemostasis valve that prevents leakage of pressurized blood. Generally, during use a distal end of the sheath 10 is passed through the skin of the subject and the sheath 10 is inserted into a vessel, such as the femoral artery. The delivery apparatus 110 (with its implant) is then inserted into the sheath 10 through the hemostasis valve, and advanced through the subject's vasculature where the implant is delivered and implanted within the subject.

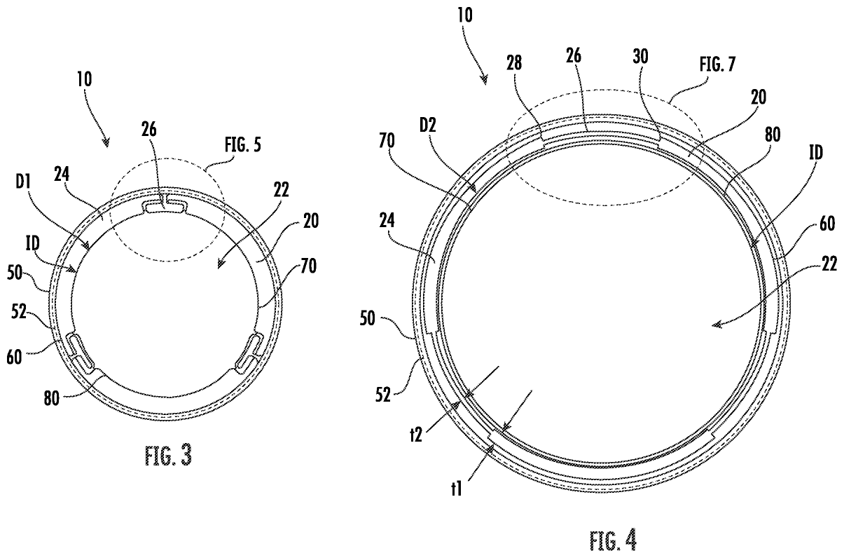

The expandable introducer sheath 10 is configured to locally transition/expand between an unexpanded and expanded configuration in response to the radially outward force of the implant/delivery apparatus 110 as it is inserted through the sheath 10. FIGS. 3 and 4 provide cross sectional views of the sheath 10 in an unexpanded and expanded configuration. In one embodiment, the sheath 10 includes an elongate inner member 20 and an elastomeric outer member 50 extending over the inner member 20 along a common central longitudinal axis. The elongated inner member 20 includes alternating portions of thin and thick wall thickness, facilitating radial expansion of the sheath 10 and providing an inner member 20 with an overall reduced wall thickness and improved column strength. In particular, the inner member 20 defines a central lumen 22, a first circumferential portion 24 (i.e., the thick wall portion) and a second circumferential portion 26 (i.e., the thin wall/folded portion in FIG. 2). The first circumferential portion 24 includes first and second longitudinal edges 28, 30 and the second circumferential portion 26 extending therebetween. As illustrated in FIGS. 3 and 4, the first circumferential portion 24 has a wall thickness (t₁) greater than a wall thickness of the second circumferential portion (t₂). The inner member 20 transitions from the unexpanded configuration (FIG. 3) to the expanded configuration (FIG. 4) in response to the radially outward force on the inner surface of the central lumen 22 by the passing implant/delivery apparatus 110.

As provided in FIG. 4, the expanded diameter (D₂) of the central lumen 22 of the elongated inner member 20 is greater than an unexpanded diameter (D₁) of the central lumen 22 of the elongated inner member 20 (FIG. 3). The unexpanded diameter ranges between about 6 mm and about 9 mm. In some embodiments, the unexpanded inner diameter is about 14F (i.e., about 0.187″). The expanded inner diameter ranges between about 6 mm and about 9 mm. For example, in some embodiments, the expanded inner diameter ranges between 24F and 26F. (i.e., about 0.325″).

The inner member 20 can include a plurality of first circumferential portions 24 and a plurality of second circumferential portions 26 extending between the first and second longitudinal edges of the adjacent first circumferential portions 24. As provided in FIGS. 3 and 4, the inner member 20 includes three first circumferential portions 24 (i.e., thick wall portions) and three second circumferential portions 26 (i.e., thin wall portions) extending between the first and second longitudinal edges of the adjacent first circumferential portions 24.

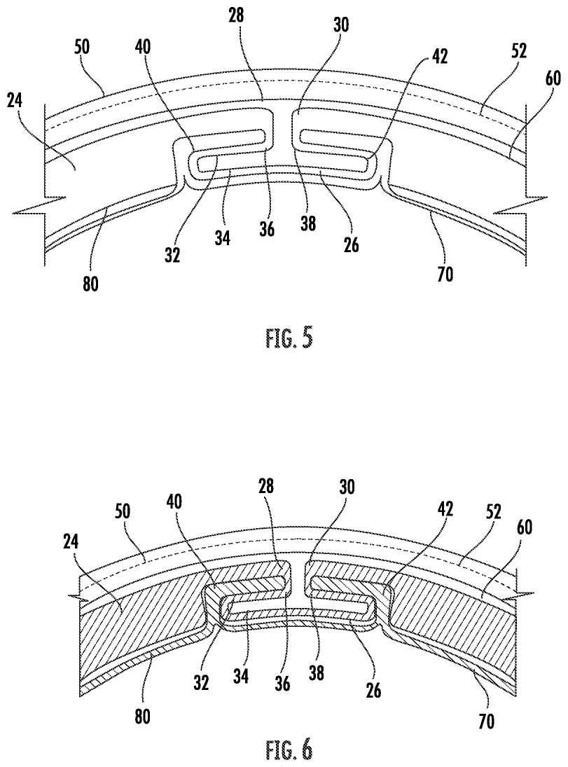

FIGS. 5 and 6 illustrate close up partial cross section views of the inner member 20 in the unexpanded/folded configuration. As illustrated in FIGS. 3, 5 and 6, in the unexpanded configuration, the elongated inner member 20 is creased proximate the first and second longitudinal edges 28, 30 into an inwardly folded configuration. In particular, when in the unexpanded/folded configuration, the second circumferential portion 26 is inwardly folded with respect to the first circumferential portion 24/outer diameter of the inner member 20. When folded, the second circumferential portion 26 includes an overlapping portion 32 and an underlying portion 34, the overlapping portion 32 positioned radially outward from the underlapping portion 34. When the elongated inner member 20 transitions between the unexpanded configuration and the expanded configuration, the overlapping portion 32 slides along the underlying portion 34 such that an amount of overlap between the overlapping and underlying portions 32, 34 decreases. Alternatively, the amount of overlap between the overlapping and underlying portions 32, 34 increases when the elongated inner member 20 transitions between the expanded and unexpanded configuration. As such, smooth and even expansion of the second circumferential portion 26/inner member 20 is achieved.

In the unexpanded/folded configuration, the second circumferential portion 26 includes a first crease 36 proximate the first longitudinal edge 28 and a second crease 38 proximate the second longitudinal edge 30. A third crease 40 is provided between the first and second creases 36, 38, and a fourth crease 42 between the third and second creases 40, 38. As such, the second circumferential portion forms an S-shaped fold between first crease 36 and the third crease 40 and a second S-shaped fold between the second crease 38 and the fourth crease 42. During expansion, the first crease 36 and the third crease 40 move closer together, and the second crease 38 and the fourth crease 42 move closer together.

As provided in FIGS. 3, 5 and 6, in the unexpanded/folded configuration, the inwardly folded second circumferential portion 26 does not project into the central lumen 22 of the inner member 20. In some examples, the inner diameter of the underlying portion 34 corresponds to the inner diameter of the first circumferential portion 24. In other examples, the inner diameter of the underlying portion 34 is less than an inner diameter of the first circumferential portion 24. In some examples, when in the folded/unexpanded configuration, a portion of the second circumferential portion 26 adjacent the first and second longitudinal edges 28, 30 extends over a portion of the inner circumference/surface of the first circumferential portion 24. In some examples, the first circumferential portion 24 has a constant wall thickness around the circumference of the inner member 20. In further examples, the first circumferential portion 24 has a reduced wall thickness adjacent the first and second longitudinal edges 28, 30, and a portion of the folded second circumferential portion 26 extends over the reduced wall thickness portion, when the sheath is in the folded/unexpanded configuration. As such, the inner diameter of the first circumferential portion 26, when folded, is equal to or less than the diameter of the first circumferential portion 24 and does not extend into the central lumen 22 of the sheath inner member 20.

In the unexpanded/folded configuration, the first circumferential portion 24 defines the main circumferential profile and provides the column strength of the sheath 10. Because the second circumferential portion 26 is inwardly folded, when the sheath 10 expands and/or during return to the unexpanded/folded configuration, the second circumferential portion 26 does not project outward of the first circumferential portion 24. As such, there are no protrusions that will interfere with and/or damage adjacent anatomy. During expansion, the second circumferential portion 26 locally unfolds into a circumferentially extended position increasing the diameter of the central lumen 22 of the inner member 20. Because expansion occurs locally, adjacent the passing implant/delivery apparatus, trauma caused to the adjacent vessel wall is reduced as the vessel is not required to maintain an expanded state during the length of the procedure. Moreover, the use of alternating (thick) first circumferential portions 24 and (thin) second circumferential portions 26 provide a sheath 10/inner member 20 with improved stiffness and column strength, reduced wall thickness and a larger expended/unfolded diameter compared to traditional expandable sheaths.

As described above, first circumferential portion 24 has a wall thickness (t₁) greater than a wall thickness (t₂) of the second circumferential portion 26. For example, the wall thickness (t₁) of the first circumferential portion 24 ranges between about 0.009″ and about 0.015″. In certain examples, the wall thickness (t₁) of the first circumferential portion 24 is about 0.011″, about 0.012″, or about 0.013″. The wall thickness (t₂) of the second circumferential portion 26 ranges between about 0.003″ and about 0.006″. In certain examples, the wall thickness (t₂) of the second circumferential portion 26 is about 0.003″, about 0.004″, or about 0.005″.

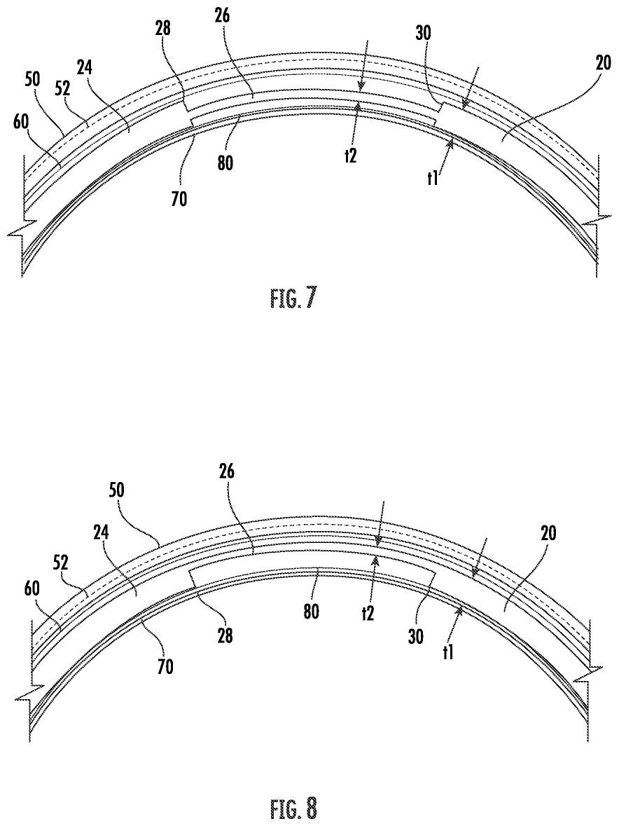

FIG. 7 shows an enlarged a partial cross-section view of the expandable introducer sheath of FIG. 4. FIGS. 8 and 9 show enlarged partial cross-section views of the expandable introducer sheath of FIG. 4 according to another embodiment. As illustrated in FIG. 7, the second circumferential portion 26 is centered with respect to the wall thickness of the first circumferential portion 24. As such, when the elongated inner member 20 is in the expanded configuration, an outer diameter of the first circumferential portion 24 is greater than an outer diameter of the second circumferential portion 26. According to another embodiment illustrated in FIG. 8, the second circumferential portion 26 is positioned adjacent a top/outer edge of the wall thickness of the first circumferential portion 24. In this example, when the elongated inner member 20 is in the expanded configuration, the outer diameter of the first circumferential portion 24 corresponds to the outer diameter of the second circumferential portion 26. In another embodiment illustrated in FIG. 9, the second circumferential portion 26 is positioned adjacent a bottom/inner edge of the wall thickness of the first circumferential portion 24. As in FIG. 7, in this embodiment, when the elongated inner member 20 is in the expanded configuration, the outer diameter of the first circumferential portion 24 is greater than the outer diameter of the second circumferential portion 26.

In certain example sheaths, the inner member 20 can comprise a polyolefin, polyamide, fluoropolymer, copolymers thereof or blends thereof. In still further embodiments, the polyolefin can comprise a high-density polyethylene, polypropylene, or blends thereof. In further examples, the inner member 20 can comprise a compound material. For example, the polymer layer of the inner member 20 can comprise a compound material comprising a polyolefin and a lubricious filler. It is understood that any of the polyolefins mentioned above can be used. In some exemplary embodiments, the polyolefin used in the compound material is high-density polyethylene. In yet other embodiments, the lubricious filler can be any filler that can improve the lubricity of the polymer layer and decrease its overall friction coefficient of the inner member 20. In some exemplary and unlimiting embodiments, the lubricious filler can comprise any additive that is known to reduce friction and behave as a lubricant. In such exemplary and unlimiting embodiments, the lubricious filler can comprise one or more of graphene, reduced graphene oxide, carbon black, boron nitride, silicones, talc, polytetrafluorethylene (PTFE), fluorinated ethylene propylene, and the like. In still further embodiments, the lubricious filler comprises a PTFE filler. In yet further embodiments, the PTFE filler is a powder.

In yet further embodiments, the lubricious filler can be present in any amount. In some exemplary and unlimiting embodiments, the lubricious filler can be present in an amount from about 5 wt % to about 20 wt % of a total weight of the compound material used to make the polymer layer of the inner liner. In yet further embodiments, the lubricious filler can be present in an exemplary amount of about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt %.

In still further embodiments, the inner member 20 comprising such compound materials is lubricious and can have a coefficient of friction less than about 0.5, less than about 0.4, less than about 0.3, less than about 0.2, less than about 0.1, or less than about 0.05, or even less than about 0.01. It is further understood that the inner member 20 can have a coefficient of friction having any value between any two foregoing values.

It is further understood that when the polymer inner member 20 comprises the disclosed herein compound material, the sheath can be substantially free of a separately disposed lubricant. For example, an additional lubricant between the inner member 20 and the outer member 50 may not be needed if the inner member 20 itself comprises a lubricious filler.

However, disclosed herein are also embodiments of the sheath where the inner member 20 comprises lubricious compound in its composition, and a separate lubricant 60 is still applied between various portions of the sheath 10. In such exemplary embodiments, this additional lubricant 60 applied manually, pad-printed, or sprayed can be applied between some of the portions of the inner member 20 and outer member 50 or all the portions of the inner member 20 and outer member 50. In some examples, lubricant 60 is disposed over at least a portion of the outer surface of the first circumferential portion 24 and/or a portion of the outer surface of the second circumferential portion 26. Any known in the art lubricants can be utilized. The lubricant 60 can comprise PTFE-based lubricant or a silicone-based lubricant. In certain and unlimiting embodiments, the lubricants can comprise Christo Lube supplied by ECL, or MED10/6670 or PRO-3499 supplied by Nusil, or TriboSys™ MDF supplied by Miller-Stephenson. In still further embodiments, it is understood that the amount of lubricant 60 can be easily determined by one of ordinary skill in the art.

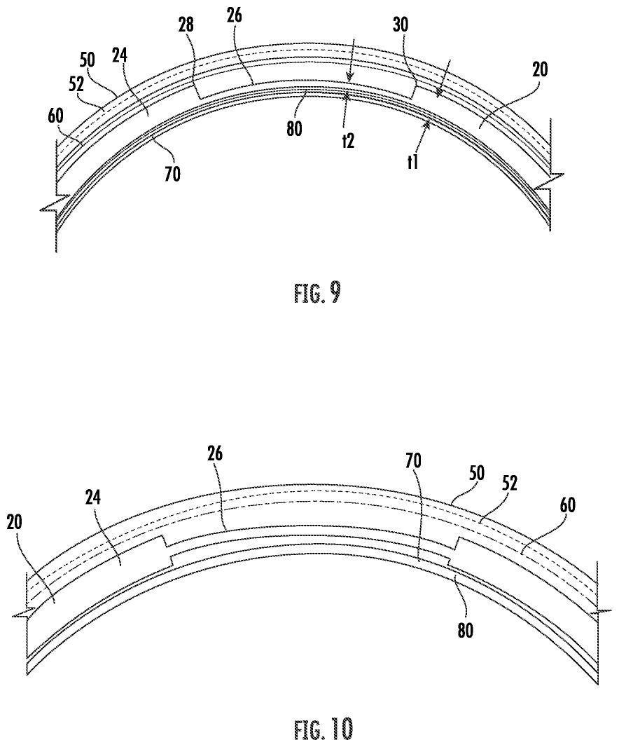

It is also understood that this additional lubricant 60 can be applied in any pattern that is desirable. For example, as illustrated in FIG. 10, the lubricant 60 is applied to the inner surface of the outer member 50 in a regular pattern around the circumference of the outer member 50. The lubricant 60 can also be applied along the entire length of the sheath or only some portions of the sheath. The lubricant 60 can also be applied in different patterns in different portions of the sheath. Yet, in other embodiments, the lubricant 60 can be applied in the same pattern along various portions of the sheath.

As described above, the lubricant 60 can also be applied by pad-printing or spraying that results in the material applied in a precisely controlled and repeatable manner, suitable for large-scale manufacturing. Detailed methods of lubricant application are discussed below.

It is understood, however, that in embodiments where the lubricant 60 is applied by pad-printing, the lubricant 60 has a viscosity prior to it is application from about 600 to about 1,200 cP, including exemplary values of about 650 cP, about 700 cP, about 750 cP, about 800 cP, about 850 cP, about 900 cP, about 950 cP, about 1,000 cP, about 1,050 cP, about 1,100 cP, and about 1150 cP.

While in embodiments where the lubricant 60 is sprayed, the lubricant 60 can have a viscosity equal to or less than about 600 cP, or about 550 cP, about 500 cP, about 450 cP, about 400 cP, about 350 cP, or equal to or less than about 350 cP.

In still further embodiments, when the lubricant 60 is cured prior to positioning the outer layer 50 on the inner member 20 of the sheath.

The lubricants can also form a film. When a film of the lubricant 60 is formed, such a film can have a thickness of equal to or less than about 20 μm, about 15 μm, about 10 μm, about 5 μm, about 1 μm, or even equal to or less than about 0.5 μm.

In some embodiments, the sheath described herein that comprises the lubricious material in the inner member 20 can exhibit a push force needed to move the prosthetic device through the sheath that is comparable or even smaller than a push force of a substantially identical reference sheath having similar configuration, wherein an inner member 20 of the substantially identical reference sheath comprises a polymer layer substantially free of a lubricious filler and comprises an amount of a lubricant disposed between the inner member 20 and the outer member 50. In other words, in some embodiments when the performances of any of the sheath configurations disclosed herein compared, in such exemplary and unlimiting embodiments, the sheath having the lubricious material in the inner member 20 and no additional lubricant present can demonstrate similar or even better performance than the similar sheath without lubricious material present in the inner member 20 but having an additional lubricant 60 dispersed between various portions of the sheath.

As described above, the sheath 10 includes an elastomeric outer member 50 extending over/around the inner member 20 along a common central longitudinal axis. The outer member 50 biases the inner member 20 towards the unexpanded/folded configuration. The outer member 50 provides an inwardly directed radial force that urges the inner member 20 towards the unexpanded/folded configuration and to return to a diameter substantially identical to the unexpanded diameter of the inner member 20. The wall thickness of the outer member 50 ranges between 0.1 mm to about 0.2 mm. In some embodiments, the wall thickness of the outer member is about 0.15 mm.

The outer member 50 is composed of an elastic material. The elasticity of the outer member 50 can be uniform or vary (longitudinally) along a length of the outer member 50. In some examples, the elasticity of the outer member increases between the proximal and distal ends of the outer member 50 such that the distal end/tip portion more easily bends to conform to the aortic geometry of a subject. Examples of an outer member having varying elasticity is further described in U.S. Provisional Patent Application No. 63/110,162 (reference no. 10836US01) titled “Flexible Catheter Devices and Methods of Manufacture and Use,” the contents of which are incorporated herein by reference in its entirety.

In some embodiments, the outer member 50 comprises an elastomeric polymer. In certain embodiments, the elastomeric polymer can comprise a polyether block amide (e.g., PEBAX®), polyamide (e.g., Vestamid®), styrene-based elastomer, polyurethane, latex, copolymers thereof, blends thereof, or co-extrudates of thereof. In certain and unlimiting embodiments, the elastomeric polymer can comprise polyether block ester copolymer, polyesters, polyvinyl chloride, thermoset silicone, poly-isoprene rubbers, polyolefin, other medical grade polymers, or combinations thereof. In yet further embodiments, the elastomeric polymer described herein can have any useful additives. In certain embodiments, the elastomeric polymers can comprise at least one friction reduction additive. In some exemplary embodiments, the friction reduction additives can comprise, for example, BaSO4, ProPell™, PTFE, any combination thereof, and the like. It is understood that this list of the friction reduction additives is not limiting, and any known in the art friction reductions additives can be utilized.

In some embodiments, the outer member 50 includes an inorganic filler. The inorganic filler can comprise bismuth oxychloride, barium sulfate, bismuth subcarbonate, calcium carbonate, aluminum trihydrate, barite, kaolin clay, limestone, or any combination thereof. While in other exemplary and unlimiting embodiments, the inorganic filler can be present in an amount of at least about 10% based on a total weight of the first compound composition. Yet, in other exemplary embodiments, the inorganic filler can be present in an amount of less than about 50% based on a total weight of the first compound composition.

In some embodiments, a solid lubricant filler is present in the outer member 50. The solid lubricant filler can comprise any additive that is known to reduce friction and behave as a lubricant. For example, the solid lubricant filler can comprise one or more of graphene, reduced graphene oxide, carbon black, boron nitride, silicones, talc, polytetrafluorethylene (PTFE), fluorinated ethylene propylene, and the like. In still further examples, the solid lubricant comprises a PTFE filler including, for example, PTFE powder. In still further embodiments, the composition of the outer member 50 can further comprise at least one tackiness reducing compound. Any compounds known in the art as capable of reducing the tackiness of the polymer composition can be considered and used for the purpose of this disclosure including, for example, ProPell™ from Foster Corporation.

It is understood that the hardness of each layer of the disclosed sheath 10 can also be varied depending on the particular application and desired properties of the sheath. In some embodiments, the outer member 50 has substantially the same durometer along the total length of the sheath 10. It is understood, however, that the durometer of the outer member 50 can also be varied along the length of the sheath 10. For example, and without limitation, disclosed herein are embodiments, where the durometer outer member 50 at a proximal end of the sheath is different from a durometer of the outer member 50 at a distal end of the sheath to provide a sheath with a more flexible distal end.

In some embodiments, the outer member 50 has a Shore A durometer between 20 A to 50 A, including exemplary values of about 25 A, about 30 A, about 35 A, about 40 A, and about 45 A. In yet further embodiments, the outer layer 50 has a Shore hardness of less than 90 Durometer, less than 80 Durometer, less than 70 Durometer, less than 60 Durometer, less than 50 Durometer, less than 40 Durometer, less than 30 Durometer, or less than 20 Durometer. In yet further exemplary embodiments, the outer member 50 has a Shore hardness from about 25 Durometer to about 75 Durometer, including exemplary values of about 30 Durometer, about 35 Durometer, about 40 Durometer, about 45 Durometer, about 50 Durometer, about 55 Durometer, about 60 Durometer, about 65 Durometer, and about 70 Durometer.

In some embodiments, the sheath 10 included a braided layer 52 which provides structural support to the sheath 10 as well as torquability to the sheath 10 during the insertion of the prosthetic device. The braided layer 52 is provided in/adjacent the outer member 50. For example, in some embodiments, the outer member 50 is a multilayer structure including the braided layer 52 and a layer of an elastomeric polymer. The braided layer 52 limits expansion of the outer member 50, and the inner member 20 accordingly, to a predetermined diameter effective to prevent ballooning of the outer member 50/sheath 10 and thereby maintain hemostasis.

The braided layer 52 extends along all or a portion of the length of the outer member 50. In some examples, the braided layer 52 is provided only along a portion of the outer member 50 adjacent the proximal end of the sheath 10. For example, the braided portion 90 can be provided along a proximal length of the sheath 10 including a tapered strain relief portion adjacent the proximal hub. The hub(s) can function as a handle for the expandable sheath assembly 22. Examples of such hubs is described in U.S. Provisional Patent Application No. 63/077,899 (titled “Reverse Bayonet Locking Hub,” filed Sep. 14, 2020), the disclosure of which is hereby incorporated by reference.

It is understood that the braided layer 52 can have any configurations known in the art. In certain embodiments, the braided layer 52 is generally a thin, hollow, substantially cylindrical tube comprising an arrangement, pattern, structure, or configuration of filaments or struts, however other geometries can also be used. The braided layer 52 includes a plurality of fibers and/or filaments interwoven into a braided pattern and defines the tubular structure. The interwoven fibers can be oriented in a single strand braid pattern or in a double strand braid pattern where each interweave includes a plurality of parallel fibers for each weave strand. In some examples, the plurality of interwoven fibers is composed of 16 individual fibers. But in other examples the plurality of interwoven fibers is composed of about 10 to about 40 individual fibers, or about 16 to about 32 individual fibers.

Each of the fibers has a curvilinear cross section, although in other examples, the fibers/filaments have a rectilinear cross section. For example, the fiber/filament is a round filament or a flat filament. Suitable filaments can be round, having a diameter less than about 0.015″, less than about 0.01″, less than about 0.008″, less than about 0.005″, less than about 0.002″, less than about 0.001″, less than about 0.0008″, or less than about 0.0005″. In yet other embodiments, suitable filaments can be round and having a diameter ranging from about 0.0005″ inches thick to about 0.015″ thick, including exemplary values of about 0.0006″, about 0.0007″, about 0.0008″, about 0.0009″, about 0.001″, about 0.002″, about 0.003″, about 0.004″, about 0.005″, about 0.006″, about 0.007″, about 0.008″, about 0.009″, about 0.01″, about 0.012″, about 0.013″, and about 0.014″. In yet other embodiments, the suitable filaments can be flat filaments having a height of less than about 0.006″, less than about 0.005″, less than about 0.004″, less than about 0.003″, less than about 0.001″, less than about 0.0009″, less than about 0.0008″, less than about 0.0007″, less than about 0.0006″, and about 0.0005″. In yet other embodiments, the flat filaments can have a width from greater than about 0.003″ to about 0.015″, including exemplary values of about 0.004″, about 0.005″, about 0.006″, about 0.007″, about 0.008″, about 0.009″, about 0.01″, about 0.012″, about 0.013″, and about 0.014″. However, other geometries and sizes are also suitable for certain embodiments.

In yet further embodiments, the braided layer 52 can have a per inch crosses (PIC) count of less than 50, less than 40, less than 30, less than 20, or less than 10. In yet other embodiments, the braid can have the PIC count from 10 to 2, including exemplary values of 9, 8, 7, 6, 5, 4, and 3. In still further embodiments, the PIC can vary along the longitudinal axis of the lumen. In yet other embodiments, the braid pattern can vary along the longitudinal axis of the lumen. In the embodiments where the braided layer 52 comprises filament that is nitinol, the nitinol is a heat-set at the expanded diameter de. In yet further embodiments, where the filament comprises stainless steel or nitinol, the filament is configured to be atraumatic at least at the distal end of the sheath.

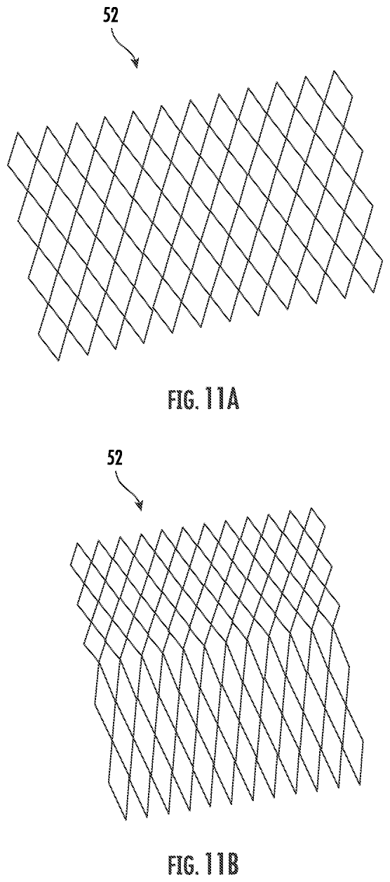

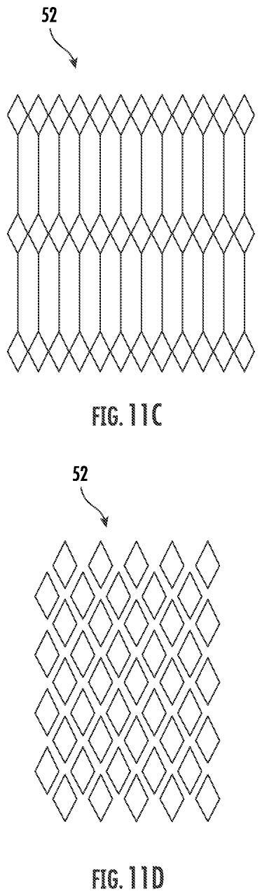

The plurality of fibers/filaments can be disposed in a plurality of circumferential rows, wherein each row has a has a sinusoidal form or any irregular form, or any combination thereof. FIGS. 11A-11D illustrate partial elevation views of various structures for the braided layer 52. It is understood that the structure, PIC, and density of the braided layer 52 can vary from section to section, changing along the length of the sheath 10. For example, the braided layer 52 can have uniform or varied braid density along the length of the sheath 10. A varied braid density can allow for omnidirectional bending with respect to the central axis of the sheath 10 because as the braid density increases, the braided layer 52 becomes more resistant to bending and axial compression. In an example sheath 10, the braid density of the braided layer 52 along the main body portion of the outer member 50 is less than the braid density of the braided layer 52 along the distal tip portion of the outer member 50. This allows the distal tip portion to resist axial compression while conforming to the vascular structure of a subject, while the body portion is rigid enough to be manipulated by a physician. It is further understood that the structures shown in FIGS. 11A-11D are not necessarily drawn to scale and show just exemplary and unlimiting embodiments.

In certain examples, the braided layer 52 can be an expandable/elastic braided structure. In yet further examples, the braided layer 52 can comprise at least one filament comprising stainless steel, nitinol, a polymer material, or a composite material. In certain unlimiting examples, the braided layer 52 comprises filaments comprising Nitinol and/or other shape memory alloys. In yet other unlimiting examples, the braided layer 52 can have filaments comprising polyester or nylon. In yet other examples, the braided layer 52 can comprise filaments comprising spectra fiber, polyethylene fiber, aramid fiber, or combinations thereof.

As illustrated in FIGS. 3 and 4, the sheath 10 includes a liner 70 provided within the central lumen 22 of the elongated inner member 20. The liner 70 provides a smooth and lubricious inner surface of the sheath 10 to facilitate passage of the implant/delivery apparatus 110. As will be described in more detail below, the liner 70 can be provided on the inner surface of the central lumen of the elongated inner member 20. An inner surface of the liner 70 defines an inner lumen of the sheath 10. In some embodiments, the lubricious liner 70 is composed of polytetrafluoroethylene (PTFE), Polyamide 12 materials, polyether block amide (PEBA), fluorinated ethylene propylene (FEP), or any other polymer suitable for forming an inner most layer of a flexible delivery sheath 10. While in other embodiments, the at least one lubricious liner 70 is at least partially etched. In general, the liner 70 extends along an entire length of the inner surface of the inner member 20/sheath 10. In some examples, the liner 70 extends along only a portion of the entire length of the inner member 20/sheath 10. The liner 70 has a thickness from about 0.001″ to about 0.005″. In some examples, the thickness of the liner 70 is about 0.003″.

The liner 70 is bonded to at least a portion of the inner surface of the inner member 20. The bonding can be done by any methods known in the art. In some embodiments, the bonding is done by heat processing, laser welding, compression bonding, and/or selective ultrasonic welding. In some examples, the liner 70 is bonded to a least a portion of the inner surface of the inner member 20 by reflow processing. In still further examples, the portion of the liner 70 bonded to the inner surface of the inner member 20 does not comprise a lubricant or a lubricant liner prior to bonding, or the portion of the liner bonded to the inner member 20 does not comprise a tie layer and/or a lubricant liner prior to bonding.

In some examples, a majority of the inner surface of the elongated inner member 20 is bonded to the liner 70. In further examples, the liner 70 is bonded to the inner surface of the inner member 20 along the first circumferential portion 24 while not bonded to the inner surface of the inner member 20 along the second circumferential portion 26. For example, as illustrated n FIG. 7, when the sheath 10 is in an expanded configuration, the liner 70 is not bonded along the second circumferential portion 26. In further examples, the liner 70 is bonded to the inner surface of the elongated inner member 70 along at least a portion of both the first and second circumferential portions 24, 26.

In some examples, as illustrated in FIG. 5, the liner 70 does not substantially extend between the first and second circumferential portions 24, 26 when the inner member 20 is in the unexpanded configuration. In further examples, as provided in FIG. 6, the liner 70 is folded with the second circumferential portion 26 and is disposed on the inner surface of the second circumferential portion 26 between the overlapping and underlying portions 32, 34 when the inner member 20 is in the unexpanded/folded configuration.

In some examples, the portion of the inner surface of the inner member 20 bonded to the liner 70 extends longitudinally along a length of the sheath 10. For example, the bond between the inner member 20 and the liner 70 is provided in longitudinally extending strips along a length of the sheath 10. In further examples, the liner 70 is bonded to the inner member 20 along a plurality of longitudinally extending strips.

The sheath 10 can also include a tie layer 80 between the elongated inner member 20 and the liner 70. The tie layer 80 is provided on an inner surface of inner member 20 and creates a surface for improved adhesion between the inner layer 20 and the liner 70. The tie layer 80 extends along a length of the sheath 10 corresponding to the liner 70. For example, if the liner 70 extends along an entire length of the inner surface of the inner member 20, the tie layer also extends along the entire length of the inner surface of the inner member 20. In other examples, where the tie layer 80 extends only along a portion of the inner surface of the inner member 20, the liner extends along the portion of the inner surface of the inner member 20. In some examples, the tie layer 80 is coextruded with the inner member 20 and/or liner 70. The tie layer 70 has a thickness from about 0.0015″ to about 0.0025″. The tie layer 70 has a thickness of about 0.002″. The tie layer 70 has an expanded (inner) diameter from about 0.244″ to about 0.406″. In some examples, the tie layer 70 has an expanded (inner) diameter of about 0.325″, corresponding to the expanded inner diameter of the inner member 20.

In yet further embodiments, the tie layer 70 can comprise any material suitable for the desired application having adhesive or bonding properties. In certain embodiment, the tie layer 70 can comprise a polyurethane material such as Tecoflex, or polymer, copolymer, or terpolymer such as maleic anhydride modified polyolefin, for example, and without limitation, Orevac® (commercially available from Arkema), ethylene acrylic acid copolymers, such as DOW Chemical Primacor®, ethylene acrylate copolymers such as Lotryl® (commercially available from Arkema), ethylene glycidyl methacrylate copolymer, ethylene acrylic esters glycidyl methacrylate terpolymer such as Lotader® (commercially available from Arkema), ethylene acrylic esters maleic anhydride terpolymers such as Lotader® or Orevac® (commercially available from Arkema).

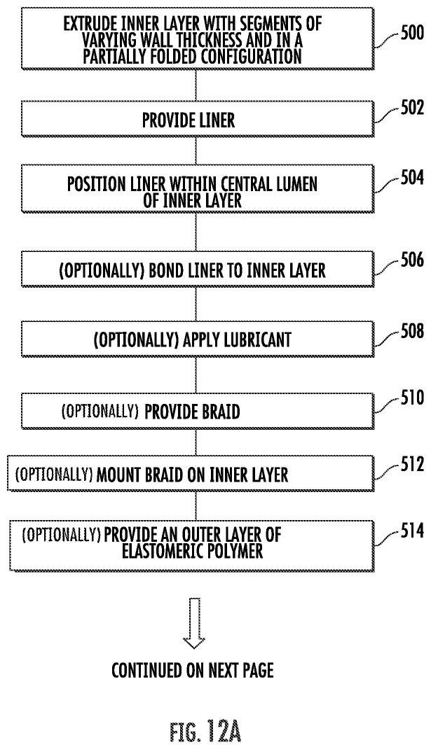

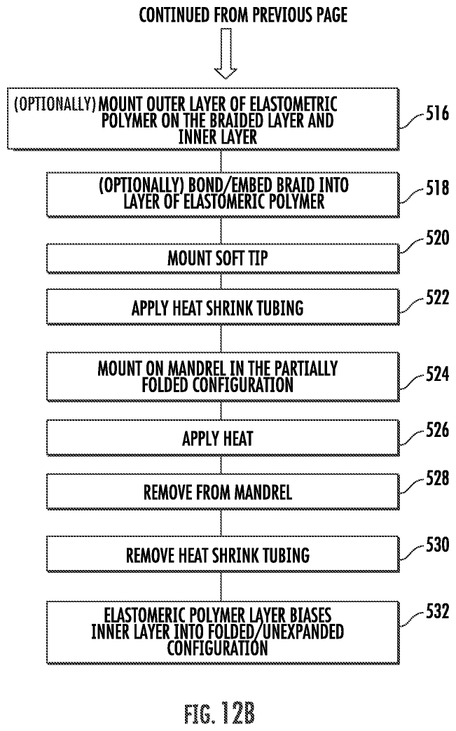

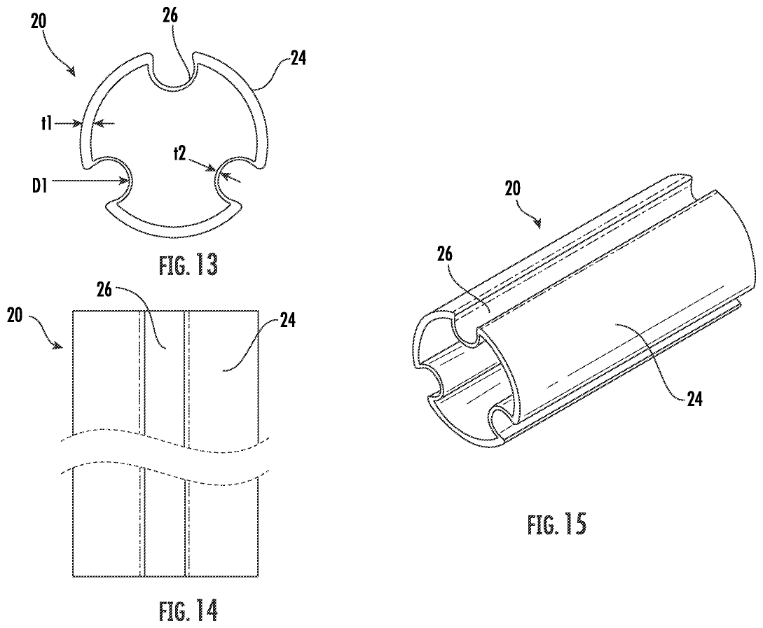

Also disclosed herein are methods of making the sheath 10. For example, disclosed herein is a method of making a sheath 10 having radially inward folded thin wall portions and adjacent (non-folding) thick wall portions. FIGS. 12A and 12B provide a block diagram of exemplary methods of producing the sheath 10 as described herein. Such methods comprising forming a variable thickness inner member 20 (Step 500). The inner member 20 is extruded and/or formed as described above, including a first circumferential portion 24 with first and second longitudinal edges 28, 30, and a second circumferential portion 26 extending between the first and second longitudinal edges 28, 30. The wall thickness of the first circumferential portion 24 being greater than the wall thickness of the second circumferential portion. As illustrated in FIGS. 3-4, the inner member 20 can include multiple, alternating, sections of varying wall thickness. FIGS. 13-15 illustrate the inner member 20 immediately after extrusion/forming and before additional processing. As provided in FIG. 13, the second circumferential portion 26 is provided as an inwardly projecting form extending toward the longitudinal axis of the inner member 20. In an example method, the second circumferential portion 26 form defines an inwardly projecting curvilinear shape in cross-section. For example, the second circumferential portion 26 form defines a semi-circular shape in cross-section. In a further example, the second circumferential portion 26 form defines a semi-circular shape in cross section having a diameter (D₁) between about 0.0375″ and about 0.625″. In yet a further example, the form diameter (D₁) is about 0.050″. The inwardly projecting form of the second circumferential portion 26 ensures that the folded shape of the final inner member 20 will include inwardly projecting/extending folds as described above.

The extruded wall thicknesses of the first and second circumferential portions 24, 26 generally correspond to the wall thicknesses of the first and second circumferential portions 24, 26 in the finished sheath. As described above, first circumferential portion 24 form has a wall thickness (t₁) greater than the wall thickness (t₂) of the second circumferential portion 26. For example, the wall thickness (t₁) of the first circumferential portion 24 form ranges between about 0.009″ and about 0.015″. In certain examples, the wall thickness (t₁) of the first circumferential portion 24 form is about 0.010″, about 0.011″, about 0.012″, about 0.013″. The wall thickness (t₂) of the second circumferential portion 26 form ranges between about 0.003″ and about 0.006″. In certain examples, the wall thickness (t₂) of the second circumferential portion 26 form is about 0.003″, about 0.004″, about 0.005″.

It is understood that the inner member 20 can be extruded or coextruded from any polymers or compounds disclosed above. For example, and without limitation, the elongated single lumen tubing of the inner member 20 can comprise at least one polymer comprising a polyolefin, a polyamide, a fluoropolymer, copolymers thereof, co-extrudates thereof, or blends thereof. Yet in other embodiments, a compound material comprising a polyolefin and a lubricious filler. In such exemplary embodiments, the polyolefin can be high-density polyethylene. Yet, in other embodiments, the lubricous filler can comprise a polytetrafluoroethylene (PTFE) filler. In such embodiments, the lubricious filler can be present in an amount from about 5 wt % to about 20 wt % of a total weight of the compound material.

For example, and without limitations, the tubular body of the inner member 20 can be extruded to form an elongated tubing comprising a compound material. This the compound material can comprise a polyolefin present in an amount from greater than 0 wt % to less than 100 wt % based on a total weight of the compound and a lubricious filler present in an amount from about 5 wt % to about 20 wt % of a total weight to the compound material.

The extruded or coextruded elongated single lumen tubing of the inner member 20 can a coefficient of friction less than about 0.5.

An inner liner 70 can be included to provide a smooth and lubricious inner surface of the sheath 10 (Step 502). In certain embodiments, and as shown in FIG. 12A, an inner liner 70 can be formed from an extruded tube having an inner surface and outer surface and having any thickness that is described above. The inner surface of the inner member 20 and/or outer surface of the liner 70 can be surface treated, such as, for example, by plasma etching, chemical etching, or other suitable methods of surface treatment. In some exemplary embodiments, where the outer surface of the liner 70 is treated, the treatment can provide for better bonding with the inner layer 20. In yet other embodiments, the inner surface of the liner 70 can be ribbed. In such exemplary embodiments, the ribbed surface facilitates a reduction of contact points with the prosthetic device and can reduce friction. It is understood that one of ordinary skill in the art can choose the composition of the inner liner 70 depending on the desired application. In certain embodiments, the decision to use a specific material for the inner liner 70 can be dependent on the desired stiffness, wall-thickness, and lubricious optimization.

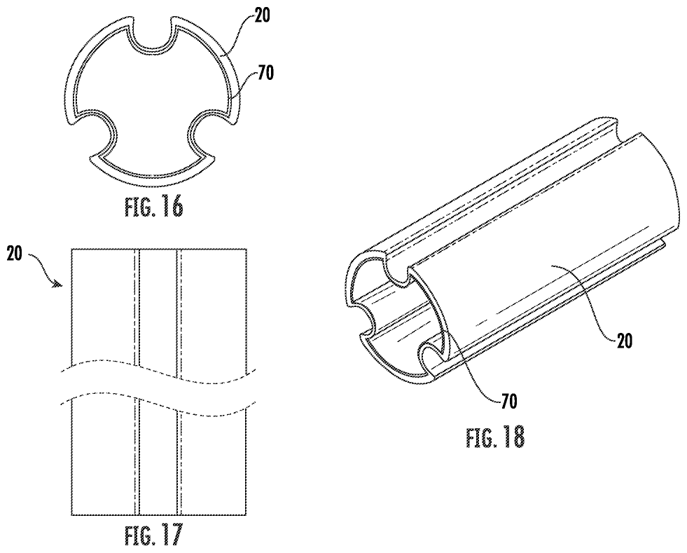

The inner liner 70 is provided within the central lumen 22 of the inner member 20 (Step 504). The liner 70 can be inserted within the central lumen 22. In other examples, the single lumen tubing of the inner member 20 can be produced by co-extrusion with multiple layers of the same or different polymers as described herein, such as liner 70. In further embodiments and as disclosed above, this tubing can be coextruded with any of the disclosed above tie layers 80. FIGS. 16-18 illustrate the inner member 20 with the liner 70 received therein, before additional processing. As provided in FIGS. 16 and 18, the liner 70 conforms to the cross-sectional shape of the inner member 20. For example, like the inner member 20, the liner 70 includes inwardly projecting forms corresponding in shape to the second circumferential portion 26, extending toward the longitudinal axis of the inner member 20. In an example method, a portion of the liner 70 defines an inwardly projecting curvilinear shape in cross section, e.g., a semi-circular shape.

In such exemplary embodiments, where for example, the tie layer 80 is present, the elongated tubing of the inner member 20 can comprise any of the disclosed above polymers, and the tie layer 80 disposed on the inner surface of the tubing or/and an outer surface of the tubing. As described above, the liner 70 is a lubricious liner can be disposed on the inner surface of the inner member 20 tubing. It is understood that this lubricious liner 70 can be disposed on the tie layer 80 if it is present. In certain aspects, the tie layer 80 is used to bond the lubricious liner 70 with the inner layer 20 forming the elongated tubing.

The liner 70 can be separately bonded to the inner member 20 (Step 506). Alternatively, the liner 70 can be bonded to the inner member 20 during a final heat treatment/reflow process (Step 526). When separately bonded (as in Step 506), the inner member 20/liner 70 are positioned on a mandrel configured to rotate. Heat processing, laser welding, compression bonding, and/or selective ultrasonic welding are applied to the inner member 20 to melt and/or fuse the inner member 20 to the liner 70. For example, laser welding uses a focused laser beam to heat the inner member 20 and liner 70 at a selected location. The mandrel is aligned with a laser beam configured to move along a longitudinal axis of the sheath to a predetermined distance at conditions effective to form the bond. The mandrel can be rotated/moved align the laser with any desired bonding location along the sheath 10. In another example, compression bonding applies pressure (and optionally heat) to select portions of the inner member 20 fusing the inner member 20 to the liner 70. In a further example, ultrasonic welding bonds the inner member 20 and liner 70 together using vibrational energy applied by a movable welding horn. The energy is absorbed and melts the inner member 20 and liner 70. Bonding methods described herein, such as ultrasonic welding, can be used prior to forming a fully assembled sheath 10.

It is understood that the specific bond and its location can be controlled as desired. For example, the temperature of the bonding device can be determined to ensure that it is set to a temperature that effective to at least partially melt both the inner member 20 and liner 70 components. In yet further embodiments, the compressive force exerted onto the part can also be adjusted such that at least partial melting is obtained without any substantial damage to the remaining components of the sheath 10. Further, a length of time the dies are compressed onto the sheath can also impact the degree of melting of the components and strength of the bond. In addition to securing the position of the inner member 20/liner 70 with respect to the bonding device, the mandrel also prevents compression of the sheath while under the load of the bonding device.

In exemplary embodiments, a lubricant 60 can be optionally applied on the outer surface of the inner member 20 (Step 508). The presence of this lubricant material can reduce the friction between the inner member 20 and the outer member 50. Similarly, lubricant material applied to the outer surface of the second circumferential portion 26 can reduce friction between the overlapping and underlying portions 32, 34 of the sheath 10 during expansion.

In still further embodiments, the methods can optionally comprise a step of providing a reinforcing member, e.g., braided layer 52 (Step 510). It is understood that any of the described above braids can be used in this step. When used, the braided layer 52 is mounted on the inner member 20 (Step 512). In some exemplary embodiment, and as shown in FIGS. 7-10, the braided layer 52 can be mounted on the lubricant 60 that can be present on the outer surface of the inner member 20. It is understood that in some embodiments, the lubricant 60 can be present only at a portion of the outer surface of the inner member 20. In these embodiments, the disclosed sheath 10 can have segments where the lubricant 60 is present and the braided layer 52 is mounted over it, while having other segments where the lubricant 60 is not present and the braided layer 52 is mounted directly on the outer surface of the inner member 20. The location of these specific segments (those without lubricant 60) can be determined by one of ordinary skill in the art depending on the desired application. It is understood that the mounting of the braided layer 52 can be done by any known in the art methods. For example, the braided layer 52 is provided as a cylindrical tube, and it can be slid on top of the inner member 20 or the first lubricant 60, if present.

The method can further optionally comprise a step of providing an outer layer of the elastomeric polymer, elastomeric outer member 50 (Step 514). Elastomeric polymers as described above can be used. The specific polymer can be chosen based on the desired properties of the disclosed sheath, such as for example, level of stiffness, hemostasis, and the like. The layer of the elastomeric polymer can be provided in any form known in the art. In certain and unlimiting embodiments, the elastomeric outer member 50 can be provided as a cylindrical tube. In still further embodiment, the elastomeric outer member 50 can be mounted on the inner member 20 and the braided layer 52 (when used) (Step 516). FIG. 10, for example, depicts an embodiment where the cylindrical tube of the elastomeric outer member 50 was slid on the inner member 20 having a lubricant 60 overlaying the inner members 20 outer surface and the braided layer 52, and subsequently heat treated.

In yet further embodiments, when the braided layer 52 is used the disclosed method can comprise a step of bonding/embedding the braided layer 52 into the layer of the elastomeric polymer, outer member 50 (Step 518). It is understood that the sheath 10 can comprise various segments. In some embodiments, some of the segments can comprise the braided layer 52 embedded within the layer of the elastomeric outer member 50, while in other segments, the braided layer 52 and the elastomeric outer member 50 are separate. It is further understood that in some embodiments, the sheath 10 can have a braided layer 52 embedded within the elastomeric outer member 50 over the whole length of the sheath 10, while in other embodiments, the braided layer 52 is not embedded within the elastomeric outer member 50 over the whole length of the sheath 10. It is further understood that any methods known in the art can be used to embed the braided layer 52 within the elastomeric outer member 50. In some embodiment, the application of the heat can be utilized. In certain embodiments, the use of heat shrink tubing can be utilized to embed the braided layer 52 within the elastomeric outer member 50. It is understood that after the step of embedment is complete, the heat shrink tubing is removed. In yet other embodiments, the braided layer 52 can be embedded within the elastomeric outer member 50 by placing the assembly in an oven or otherwise heating it. Alternatively, the braided layer 52 can be bonded to the elastomeric outer member 50 during a final heat treatment/reflow process (Step 526).

In still further embodiments, a soft, atraumatic tip 54 (FIG. 2) can be provided at the distal end of the sheath 10 (Step 520). In some embodiments, the distal tip 54 is provided by forming a distally tapered shape into an elastomeric end portion of the inner member 20 and/or elastomeric outer member 50. For example, by heat forming a distally tapered shape at the distal end of the sheath 10. In further embodiments, the distal tip 54, including a distally tapered outer surface, is mounted to the distal end of the sheath 10, e.g., at the distal end of the elastomeric outer member 50 and/or at the distal end of the inner member 20. In some examples, the distal tip 54 is mounted by heat processing, laser welding, compression bonding, and/or selective ultrasonic welding the distal tip 54 to the elastomeric outer member 50 or the inner member 20. In other examples, the distal tip 54 is mounted to the elastomeric outer member 50 or the inner member 20 by a chemical and/or mechanical fastener. In yet further embodiments, a radiopaque marker is embedded proximate the distal end of the inner member 20 and/or distal end of the elastomeric outer member 50. In some examples, the radiopaque marker is embedded in the distal top 54.

In yet further embodiments, the elastomeric outer member 50 comprising the braided layer 52 (optional) and inner member 20 is at least partially bonded to the liner 70 are bonded together to form the sheath 10. The sheath 10 is biased in a folded/unexpanded configuration by the radially inward force provided by the elastomeric outer member 50. It is understood that this bonding can also be achieved by any known in the art methods. In certain embodiments, a heat shrink tubing is applied to the outer surface of the elastomeric outer member 50 (Step 522).

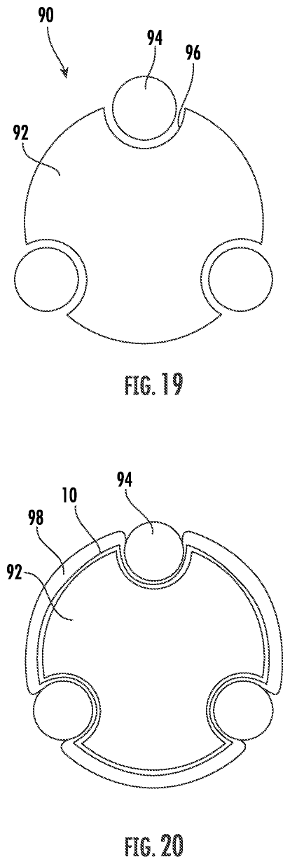

The sheath 10 is then mounted on a mandrel 90 (Step 524). An end view of an example mandrel 90 is depicted in FIG. 19. FIG. 20 illustrates the sheath 10 mounted on the mandrel 90. The mandrel 90 includes a central mandrel 92 having size and shape corresponding to the cross-sectional profile of the inner member 20 form as originally extruded, and a secondary mandrel 94 located adjacent the perimeter of the central mandrel 92. As provided in FIG. 20, the central mandrel 92 includes a recess 96 having a size, shape and location corresponding to the second circumferential portion 26 form of the extruded inner member 20. The central mandrel 92 the number of recesses 96 corresponding to the number of second circumferential portions 26 included on the inner member 20. The secondary mandrel 94 is positioned at least partially within the recess 96 formed in the central mandrel 92.

FIG. 20 illustrates the sheath 10 with the heat shrink tubing 98 mounted on the mandrel 90. As provided in FIG. 20, the central mandrel 92 has a size and shape corresponding to the cross-sectional profile of the sheath 10/inner member 20 as originally extruded. When mounted, the secondary mandrels 94 maintain the outer profile of the sheath 10. In particular, the secondary mandrels 94 maintain the inwardly projecting shape of the second circumferential portion 26, while the central mandrel 92 also maintains the uniform circumferential shape of the first circumferential portion 24.

The various layers of the sheath 10 are then bonded together using heat processing (Step 526). In some embodiments, heat process bonding of the sheath 10 layers is achieved by placing the assembly in an oven or otherwise heating it. In other embodiments, the bonding is performed by heating at a temperature from about 350° F. to about 550° F. for a time period effective to form a bond between at least a portion of the elastomeric outer member 50 and at least a portion of one of the braided layer 52, inner member 20 and/or the inner liner 70. In yet further embodiments, the heating can be done at a temperature of about 375° F., about 400° F., about 425° F., about 450° F., about 475° F., about 500° F., or about 525° F. In yet other embodiments, the time period effective to form a bond can comprise from about 1 second to about 60 seconds, including exemplary values of about 5 seconds, about 10 seconds, about 15 seconds, about 20 seconds, about 25 seconds, about 30 seconds, about 35 seconds, about 40 seconds, about 45 seconds, about 50 seconds, and about 55 seconds. However, it is further understood that this time period is not limiting, and it can have any value needed to provide for an effective bond, for example, it can have any value from about 1 second to about 5 hours.

Once the heating process is complete, the sheath 10 is removed from the mandrel (Step 528) and the heat shrink tubing (if used) is removed and the proximal end hub is attached to the sheath (Step 530).

With the heat processing complete and the sheath 10 removed from the mandrel 90, the inner member 20 is biased in the unexpanded/folded configuration by the radially inward force of the elastomeric outer member 50 (Step 532). As described above, because the second circumferential portions 26 are pre-formed having an inwardly projecting form, the second circumferential portion 25 is biased to fold inward with respect to the outer diameter of the elastomeric outer member 50.

Also disclosed herein are methods of deploying a prosthetic into a subject using sheath 10. The method includes positioning an expandable sheath 10 within the vascular system of a subject. A prosthetic device, such as a prosthetic heart valve, is introduced into a lumen of the expandable sheath 10 and advanced through the lumen of the sheath 10 such that the prosthetic device exerts a radially outward force on an inner surface of an inner member 20 of the sheath 10 and locally unfolds the inner member 10 into an expanded configuration.

Introducer sheaths and delivery devices may be utilized in a variety of subjects and procedures. Subjects include (but are not limited to) medical patients, veterinary patients, animal models, cadavers, and simulators of the cardiac and vasculature system (e.g., anthropomorphic phantoms and explant tissue). Procedures include (but are not limited to) medical and training procedures.

The prosthetic device is advanced through the distal tip portion 54 of the sheath 10, locally enlarging the lumen of the distal tip portion in response to radial pressure exerted by passage of the prosthetic device.

Radially inward force provided the elastomeric outer member 50 at least partially collapses/returns to the inner member and the distal tip portion toward the unexpanded/folded configuration after the prosthetic device has passed.

The prosthetic device passes through the opening in the distal end of the sheath 10 and is delivered to the procedure site, e.g., an aortic valve. With the prosthetic device delivered, the sheath 10 is removed from the subject's vasculature.

It will be appreciated that embodiments of the heart valve delivery system and expandable sheath 10 described herein provide improved devices and methods for advancing a prosthetic heart valve through a subject's vasculature. Beyond transcatheter heart valves, the expandable sheath 10 can be useful for other types of minimally invasive procedure, such as any procedure requiring introduction of an apparatus into a subject's vessel. For example, the expandable sheath 10 can be used to introduce other types of delivery apparatus for placing various types of intraluminal devices (e.g., stents, stented grafts, balloon catheters for angioplasty procedures, etc.) into many types of vascular and non-vascular body lumens (e.g., veins, arteries, esophagus, ducts of the biliary tree, intestine, urethra, fallopian tube, other endocrine or exocrine ducts, etc.).

### EXEMPLARY ASPECTS

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1: An expandable sheath comprising: an elongated inner member defining a central lumen, a first circumferential portion including a first and second longitudinal edges, and a second circumferential portion extending between the first and second longitudinal edges, the first circumferential portion having a wall thickness greater than a wall thickness of the second circumferential portion; wherein the elongated inner member is configured to transition from an unexpanded configuration to an expanded configuration in response to a radially outward force on an inner surface of the central lumen; wherein, in the unexpanded configuration, the elongated inner member is creased proximate the first and second longitudinal edges into a folded configuration such that the second circumferential portion is positioned at least partially radially inward of the first circumferential portion

Example 2: The introducer according to any example herein, particularly example 1, wherein an expanded diameter of a central lumen of the elongated inner member in the expanded configuration is greater than an unexpanded diameter of the central lumen of the elongated inner member in the unexpanded configuration.

Example 3: The introducer according to any example herein, particularly example 2, wherein the expanded inner diameter ranges between about 6 mm and about 9 mm.

Example 4: The introducer according to any example herein, particularly examples 2-3, wherein the expanded inner diameter ranges between 24F and 26F. (i.e., about 0.325″).

Example 5: The introducer according to any example herein, particularly examples 2-4, wherein the unexpanded diameter ranges between about 6 mm and about 9 mm.

Example 6: The introducer according to any example herein, particularly examples 2-5, wherein the unexpanded inner diameter is about 14F (i.e., about 0.187″).

Example 7: The introducer according to any example herein, particularly examples 1-6, wherein the elongated inner member includes a plurality of first circumferential portions and a plurality of second circumferential portions extending between the first and second longitudinal edges of the adjacent first circumferential portions.

Example 8: The introducer according to any example herein, particularly example 7, wherein the elongated inner member includes three first circumferential portions and three second circumferential portions extending between the first and second longitudinal edges of the adjacent first circumferential portions.

Example 9: The introducer according to any example herein, particularly examples 1-8, wherein the elongated inner member is configured to locally expanded from the unexpanded configuration to the expanded configuration.

Example 10: The introducer according to any example herein, particularly examples 1-9, wherein, when the elongated inner member creases proximate the first and second longitudinal edges into the unexpanded configuration, the second circumferential portion is creased into a folded configuration radially inward of an outer diameter of the first circumferential portion.

Example 11: The introducer according to any example herein, particularly example 10, wherein the second circumferential portion locally unfolds in response to a radially outward force on the inner surface of the central lumen.

Example 12: The introducer according to any example herein, particularly examples 10-11, wherein, in the folded configuration, the second circumferential portion includes an overlapping portion and an underlying portion, the overlapping portion position radially outward from the underlapping portion.

Example 13: The introducer according to any example herein, particularly example 12, wherein, when the elongated inner member transitions between the unexpanded configuration and the expanded configuration, the overlapping portion slides along the underlying portion such that an amount of overlap between the overlapping an underlying portions decreases, and increases when the elongated inner member transition between the expanded and unexpanded configuration.

Example 14: The introducer according to any example herein, particularly examples 12-13, wherein, in the folded configuration, an inner diameter of the underlying portion corresponds to an inner diameter of the first circumferential portion.

Example 15: The introducer according to any example herein, particularly examples 12-13, wherein, in the folded configuration, an inner diameter of the underlying portion is less than an inner diameter of the first circumferential portion.

Example 16: The introducer according to any example herein, particularly examples 1-15, wherein, in the unexpanded configuration, the second circumferential portion includes a first crease proximate the first longitudinal edge and a second crease proximate the second longitudinal edge.

Examples 17: The introducer according to any example herein, particularly example 16, wherein, in the unexpanded configuration, the second circumferential portion includes a third crease between the first and second crease.

Example 18: The introducer according to any example herein, particularly examples 16-17, wherein, in the unexpanded configuration, the second circumferential portion includes a fourth crease between the third and second crease.

Example 19: The introducer according to any example herein, particularly examples 1-18, wherein, in the unexpanded configuration, the second circumferential portion includes at least one S-fold.

Example 20: The introducer according to any example herein, particularly examples 1-19, wherein, in the unexpanded configuration, the second circumferential portion includes two S-folds.

Example 21: The introducer according to any example herein, particularly examples 1-20, wherein the elongated inner member comprises a polymer.

Example 22: The introducer according to any example herein, particularly example 21, wherein the polymer comprises a polyolefin, a polyamide, a fluoropolymer, copolymers thereof, co-extrudates thereof, or blends thereof.

Example 23: The introducer according to any example herein, particularly example 22, wherein the polyolefin comprises a high-density polyethylene, polypropylene, or blends thereof.

Example 24: The introducer according to any example herein, particularly examples 1-23, wherein the elongated inner member comprises a compound material comprising a polyolefin and a lubricious filler.

Example 25: The introducer according to any example herein, particularly example 24, wherein the polyolefin is high-density polyethylene.

Example 26: The introducer according to any example herein, particularly examples 24 or 25, wherein the lubricous filler comprises a polytetrafluoroethylene (PTFE) filler.

Example 27: The introducer according to any example herein, particularly examples 24-26, wherein the lubricious filler is present in an amount from about 5 wt % to about 20 wt % of a total weight of the compound material.

Example 28: The introducer according to any example herein, particularly examples 24-27, wherein the sheet is lubricious and has a coefficient of friction less than about 0.5.

Example 29: The introducer according to any example herein, particularly examples 24-28, wherein the polymer layer of the sheet comprises the compound material, the sheath is substantially free of a separately disposed lubricant.

Example 30: The introducer according to any example herein, particularly examples 1-29, wherein the wall thickness of the first circumferential portion ranges between about 0.009″ and about 0.015″.

Example 31: The introducer according to any example herein, particularly example 30, wherein the wall thickness of the first circumferential portion is about 0.012″.

Example 32: The introducer according to any example herein, particularly examples 1-31, wherein the wall thickness of the second circumferential portion ranges between about 0.003″ and about 0.006″.

Example 33: The introducer according to any example herein, particularly example 32, wherein the wall thickness of the second circumferential portion is about 0.004″.

Example 34: The introducer according to any example herein, particularly examples 1-33, wherein the second circumferential portion is centered with respect to the wall thickness of the first circumferential portion.

Example 35: The introducer according to any example herein, particularly examples 1-33, wherein, when the elongated inner member is in the expanded configuration, an outer diameter of the first circumferential portion is greater than an outer diameter of the second circumferential portion.

Example 36: The introducer according to any example herein, particularly examples 1-31, wherein the second circumferential portion is positioned adjacent a top/outer edge of the wall thickness of the first circumferential portion.

Example 37: The introducer according to any example herein, particularly example 36, wherein, when the elongated inner member is in the expanded configuration, an outer diameter of the first circumferential portion corresponds to an outer diameter of the second circumferential portion.

Example 38: The introducer according to any example herein, particularly examples 1-37, further including a liner provided within a central lumen of the elongated inner member.

Example 39: The introducer according to any example herein, particularly example 38, wherein the liner is provided on an inner surface of the central lumen of the elongated inner member.

Example 40: The introducer according to any example herein, particularly examples 38-39, wherein the liner is not bonded to the inner surface of the elongated inner member.

Example 41: The introducer according to any example herein, particularly examples 38-39, wherein the liner is bonded to the elongated inner member.

Example 42: The introducer according to any example herein, particularly example 41, wherein at least a portion of an inner surface of the elongated inner member is bonded to at least a portion the liner by heat processing, laser welding, compression bonding, and/or selective ultrasonic welding

Example 43: The introducer according to any example herein, particularly example 42, wherein the at least a portion of an inner surface of the elongated inner member is bonded to the at least a portion the liner by reflow processing.

Example 44: The introducer according to any example herein, particularly examples 41-43, wherein a majority of an inner surface of the elongated inner member is bonded to the liner.

Example 45: The introducer according to any example herein, particularly examples 41-44, wherein the liner is bonded to an inner surface of the elongated inner member along the first circumferential portion and the liner is not bonded to an inner surface of the elongated inner member along the second circumferential portion.

Example 46: The introducer according to any example herein, particularly examples 41-45, wherein the liner is bonded to an inner surface of the elongated inner member along at least a portion of both the first and second circumferential portions Example xx: The introducer according to any example herein, particularly examples

Example 47: The introducer according to any example herein, particularly examples 41-46, wherein the at least a portion of an outer surface the liner that is bonded to the at least a portion of an inner surface of the elongated inner member does not comprise a lubricant or a lubricant liner prior to bonding, or wherein the at least a portion of an outer surface the liner bonded to the at least a portion of an inner surface of the elongated inner member does not comprise a tie layer and/or a lubricant liner prior to bonding.

Example 48: The introducer according to any example herein, particularly examples 41-47, wherein the at least a portion of an inner surface of the elongated inner member bonded to the at least a portion of an outer surface of the liner extends longitudinally along a length of the sheath.

Example 49: The introducer according to any example herein, particularly examples 41-48, wherein two or more portions of the inner surface of the elongated inner member bonded to two or more portions of the outer surface of the liner extends longitudinally along a length of the sheath.

Example 50: The introducer according to any example herein, particularly examples 38-49, wherein the liner is a lubricious liner.

Example 51: The introducer according to any example herein, particularly examples 38-50, wherein the liner comprises PTFE.

Example 52: The introducer according to any example herein, particularly example 51, wherein the liner is at least partially etched.

Example 53: The introducer according to any example herein, particularly examples 38-52, wherein the liner has a thickness from about 0.001″ to about 0.005″.

Example 54: The introducer according to any example herein, particularly examples 53, wherein the liner has a thickness of about 0.003″.

Example 55: The introducer according to any example herein, particularly examples 1-54, further including: an outer elastomeric member extending around the elongated inner member and configured to bias the elongated inner member towards the unexpanded configuration.

Example 56: The introducer according to any example herein, particularly example 55, wherein an expanded diameter of a central lumen of the elongated inner member is greater in the expanded configuration than an unexpanded diameter of the central lumen of the elongated inner member in the unexpanded configuration, and wherein the outer elastomeric member biases the inner member to a return diameter that is substantially identical to the unexpanded diameter.

Example 57: The introducer according to any example herein, particularly examples 55-56, wherein the outer elastomeric member comprises a polyether block amide (e.g., PEBAX®), a styrene-based elastomer, polyurethane, latex, copolymers thereof, blends thereof, or extrudates of thereof.

Example 58: The introducer according to any example herein, particularly examples 55-57, wherein the outer elastomeric member comprises a blend of the styrene-based elastomer and polyurethane.

Example 59: The introducer according to any example herein, particularly examples 55-58, wherein the styrene-based elastomer has a Shore A durometer between 20 A to 50 A.

Example 60: The introducer according to any example herein, particularly examples 55-59, wherein the outer elastomeric member includes an inorganic filler.

Example 61: The introducer according to any example herein, particularly example 60, wherein the inorganic filler comprises bismuth oxychloride, barium sulfate, bismuth subcarbonate, calcium carbonate, aluminum trihydrate, barite, kaolin clay, limestone, or any combination thereof.

Example 62: The introducer according to any example herein, particularly examples 55-61, wherein the outer elastomeric member includes a solid lubricant filler.

Example 63: The introducer according to any example herein, particularly example 62, wherein the solid lubricant comprises a PTFE filler (e.g., PTFE powder).

Example 64: The introducer according to any example herein, particularly examples 55-63, wherein the outer elastomeric member has a thickness from about 0.1 mm to about 0.2 mm.

Example 65: The introducer according to any example herein, particularly example 62, wherein the outer member has a thickness of about 0.15 mm.

Example 66: The introducer according to any example herein, particularly examples 36-65, further comprising at least one tie layer between the elongated inner member and the liner.

Example 67: The introducer according to any example herein, particularly example 66, wherein the tie layer can comprise a polyurethane, polymer, copolymer, or terpolymer, and/or combinations thereof.

Example 68: The introducer according to any example herein, particularly examples 66-67, wherein the tie layer has a thickness from about 0.0015″ to about 0.0025″.

Example 69: The introducer according to any example herein, particularly examples 66-68, wherein the tie layer has a thickness of about 0.002″.

Example 70: The introducer according to any example herein, particularly examples 66-69, wherein the tie layer has an (inner) diameter from about 0.244″ to about 0.406″.

Example 71: The introducer according to any example herein, particularly examples 66-70, wherein the tie layer has an (inner) diameter of about 0.325″.

Example 72: The introducer according to any example herein, particularly examples 55-71, wherein the outer elastomeric member is a multilayer structure.

Example 73: The introducer according to any example herein, particularly examples 1-66, wherein the elongated inner member includes a reinforcing element embedded in an elastomeric material.

Example 74: The introducer according to any example herein, particularly example 67, further including: an outer elastomeric member extending around the elongated inner member and configured to bias the elongated inner member into the folded configuration, wherein the reinforcing element is embedded in the elastomeric material of the outer elastomeric member.

Example 75: The introducer according to any example herein, particularly examples 67-68, wherein the reinforcing element limits expansion of the elongated inner member a predetermined diameter effective to prevent ballooning of the outer layer of the sheath and thereby to maintain hemostasis.

Example 76: The introducer according to any example herein, particularly examples 73-75, wherein the reinforcing element extends along a length of the sheath.

Example 77: The introducer according to any example herein, particularly example 76, wherein the reinforcing element extends along an entire length of the sheath.

Example 78: The introducer according to any example herein, particularly examples 76-77, wherein the reinforcing element extends along a length of the sheath extending from a proximal end of the sheath.

Example 79: The introducer according to any example herein, particularly example 78, wherein the length of the sheath includes a tapered strain relief portion.

Example 80: The introducer according to any example herein, particularly examples 73-79, wherein the reinforcing element comprises a plurality of filaments arranged in a braid configuration.

Example 81: The introducer according to any example herein, particularly examples 73-80, wherein the reinforcing element comprises a plurality of filaments disposed in a plurality of circumferential rows with respect to the outer member, wherein each of plurality has a sinusoidal form or any irregular form, or any combination thereof.

Example 82: The introducer according to any example herein, particularly examples 80-81, wherein the plurality of filaments comprise stainless steel, nitinol, a polymer material, or composite material.

Example 83: The introducer according to any example herein, particularly example 82, wherein the polymer material is polyester or nylon.

Example 84: The introducer according to any example herein, particularly examples 80-83, wherein the filament is a round filament or a flat filament.

Example 85: The introducer according to any example herein, particularly example 84, wherein the round filament has a diameter of less than about 0.015″.

Example 86: The introducer according to any example herein, particularly examples 84-85, wherein the flat filament has a height of less than about 0.006″ and a width from greater than about 0.003″ to about 0.015″.

Example 87: The introducer according to any example herein, particularly examples 80-76, wherein the braid has a per inch crosses (PIC) count less than 50.

Example 88: The introducer according to any example herein, particularly examples 1-87, wherein the elongated inner member further comprises an elastomeric distal tip extending from a distal end of the elongated inner member.

Example 89: The introducer according to any example herein, particularly example 88 wherein the distal tip has a distally tapering shape.

Example 90: The introducer according to any example herein, particularly examples 88-89, wherein the distal tip further comprises a marker embedded in the distal tip.

Example 91: The introducer according to any example herein, particularly examples 1-80, including a lubricant disposed over at least a portion of an outer surface of the inner member and/or an inner surface of an outer elastic member.

Example 92: The introducer according to any example herein, particularly example 91, wherein the lubricant is disposed over at least a portion of an outer surface of the first circumferential portion.

Example 93: The introducer according to any example herein, particularly example 91, wherein the lubricant is disposed over at least a portion of an outer surface of the second circumferential portion.

Example 94: The introducer according to any example herein, particularly examples 91-93, wherein the lubricant comprises PTFE-based lubricant or a silicone-based lubricant.

Example 95: The introducer according to any example herein, particularly examples 91-94, wherein the disposed lubricant has a predetermined pattern.

Example 96: The introducer according to any example herein, particularly examples 91-95, wherein the lubricant is pad-printed.

Example 97: The introducer according to any example herein, particularly examples 91-96, wherein the lubricant is sprayed.

Example 98: The introducer according to any example herein, particularly examples 91-97, wherein a viscosity of the lubricant prior to disposing is about 600 to about 1,200 cP.

Example 99: The introducer according to any example herein, particularly examples 91-98, wherein a viscosity of the lubricant prior to disposing is equal to or less than about 600 cP.

Example 100: The introducer according to any example herein, particularly examples 91-99, wherein the lubricant forms a film having a thickness of equal to or less than about 20 μm.

Example 101: The introducer according to any example herein, particularly examples 99-100, wherein the lubricant is cured.

Example 102: A method of making an expandable sheath, the method comprising: forming an inner member having portions of varying wall thickness including a first circumferential portion having a first and second longitudinal edges, and a second circumferential portion extending between the first and second longitudinal edges, the first circumferential portion having a wall thickness greater than a wall thickness of the second circumferential portion; and disposing an outer elastomeric member over the inner member to form the sheath; wherein the sheath is configured to transition from an unexpanded configuration to an expanded configuration in response to a radially outward force on an inner surface of a central lumen of the inner member, wherein the outer elastomeric member urges the inner member towards the unexpanded configuration, wherein, in the unexpanded configuration the elongated inner member is creased proximate the first and second longitudinal edges into a folded configuration such that the second circumferential portion is positioned at least partially radially inward of the first circumferential portion.

Example 103: The method of making an expandable sheath according to any example herein, particularly example 102, wherein forming an inner member includes forming the second circumferential portion to extend toward a longitudinal axis of the inner member.

Example 104: The method of making an expandable sheath according to any example herein, particularly examples 102-103, wherein forming an inner member includes forming the second circumferential portion to define an inwardly projecting curvilinear shape in cross section.

Example 105: The method of making an expandable sheath according to any example herein, particularly examples 102-104, wherein forming an inner member includes forming the second circumferential portion to define a semi-circular shape in cross section.

Example 106: The method of making an expandable sheath according to any example herein, particularly examples 102-105, wherein the inner member is formed by extrusion.

Example 107: The method of making an expandable sheath according to any example herein, particularly examples 102-106, further including: disposing a liner within a central lumen of the inner member.

Example 108: The method of making an expandable sheath according to any example herein, particularly example 107, further including: bonding the liner to an inner surface of the inner member

Example 109: The method of making an expandable sheath according to any example herein, particularly example 108, wherein at least a portion of an inner surface of the elongated inner member is bonded to at least a portion of an outer surface of the liner by heat processing, laser welding, compression bonding, and/or selective ultrasonic welding

Example 110: The method of making an expandable sheath according to any example herein, particularly example 109, wherein the at least a portion of an inner surface of the elongated inner member is bonded to the at least a portion of an outer surface of the liner by reflow processing.

Example 111: The method of making an expandable sheath according to any example herein, particularly examples 108-109, wherein a majority of an inner surface of the elongated member is bonded to a majority of an outer surface of the liner.

Example 112: The method of making an expandable sheath according to any example herein, particularly examples 108-111, wherein the liner is bonded to an inner surface of the elongated member along the first circumferential portion and the liner is not bonded to an inner surface of the elongated member along the second circumferential portion.

Example 113: The method of making an expandable sheath according to any example herein, particularly examples 108-112, wherein the liner is bonded to an inner surface of the elongated member along at least a portion of both the first and second circumferential portions.

Example 114: The method of making an expandable sheath according to any example herein, particularly examples 108-113, wherein the at least a portion of an outer surface the liner that is bonded to the at least a portion of an inner surface of the elongated inner member does not comprise a lubricant or a lubricant liner prior to bonding, or wherein the at least a portion of an outer surface the liner bonded to the at least a portion of an inner surface of the elongated inner member does not comprise a tie layer and/or a lubricant liner prior to bonding.

Example 115: The method of making an expandable sheath according to any example herein, particularly examples 108-114, wherein the at least a portion of an inner surface of the elongated inner member bonded to the at least a portion of an outer surface of the liner extends longitudinally along a length of the sheath.

Example 116: The method of making an expandable sheath according to any example herein, particularly examples 108-115, wherein two or more portions of the inner surface of the elongated inner member bonded to two or more portions of the outer surface of the liner extends longitudinally along a length of the sheath.

Example 117: The method of making an expandable sheath according to any example herein, particularly examples 102-116, further including: providing a lubricant over at least a portion of the outer surface of the inner member.

Example 118: The method of making an expandable sheath according to any example herein, particularly examples 102-117, further including: providing a reinforcing element over the inner member.

Example 119: The method of making an expandable sheath according to any example herein, particularly example 118, wherein the reinforcing element comprises a plurality of filaments arranged in a braid configuration.

Example 120: The method of making an expandable sheath according to any example herein, particularly examples 118-119, wherein the reinforcing member is bonded to the outer elastomeric member.

Example 121: The method of making an expandable sheath according to any example herein, particularly examples 118-120, wherein the reinforcing member is embedded in the outer elastomeric member.

Example 122: The method of making an expandable sheath according to any example herein, particularly examples 102-121, further comprising: forming a tapered shape into an elastomeric end portion of the elongate inner member and/or outer elastomeric member.

Example 123: The method of making an expandable sheath according to any example herein, particularly examples 102-122, further comprising: mounting an elastomeric distal tip to a distal end of the sheath

Example 124: The method of making an expandable sheath according to any example herein, particularly example 123, wherein the distal tip is mounted to the distal end of the sheath by heat processing, laser welding, compression bonding, and/or selective ultrasonic welding.

Example 125: The method of making an expandable sheath according to any example herein, particularly examples 123-124, wherein the distal tip is mounted to the distal end of the sheath by a chemical and/or mechanical fastener.

Example 126: The method of making an expandable sheath according to any example herein, particularly examples 123-125, further including: embedding a marker proximal at distal end of the inner member and/or at the distal tip.

Example 127: The method of making an expandable sheath according to any example herein, particularly examples 102-126, further comprising: applying a heat shrink layer to an outer surface of the outer elastomeric layer.

Example 128: The method of making an expandable sheath according to any example herein, particularly example 127, further comprising: mounting sheath on a mandrel.

Example 129: The method of making an expandable sheath according to any example herein, particularly example 128, wherein mounting the sheath on a mandrel includes maintaining the second circumferential portion in a configuration extending towards a longitudinal axis of the inner member.

Example 130: The method of making an expandable sheath according to any example herein, particularly examples 128-129, wherein mounting the sheath on a mandrel includes maintaining the second circumferential portion in a configuration having an inwardly projecting curvilinear shape in cross section.

Example 131: The method of making an expandable sheath according to any example herein, particularly examples 128-130, further comprising: bonding at least one of the inner member, outer elastomeric member, liner, and braid by heat processing.

Example 132: The method of making an expandable sheath according to any example herein, particularly example 131, wherein heat processing is performed by heating at a temperature from about 350° F. to about 550° F. for a time period effective to form a bond between at least a portion of at least one of the inner member, outer elastomeric member, liner, and braid (if used).

Example 133: The method of making an expandable sheath according to any example herein, particularly example 132, wherein heat processing is performed at a temperature of about 375° F., about 400° F., about 425° F., about 450° F., about 475° F., about 500° F., or about 525° F.

Example 134: The method of making an expandable sheath according to any example herein, particularly examples 131-133, wherein the heat processing occurs for a time period effective to form a bond, where the time period comprises from about 1 second to about 55 seconds.

Example 135: The method of making an expandable sheath according to any example herein, particularly examples 131-134, wherein the heat processing occurs for a time period effective to form a bond, where the time period comprises from about 1 second to about 60 seconds, including exemplary values of about 5 seconds, about 10 seconds, about 15 seconds, about 20 seconds, about 25 seconds, about 30 seconds, about 35 seconds, about 40 seconds, about 45 seconds, about 50 seconds, and about 55 seconds.

Example 136: The method of making an expandable sheath according to any example herein, particularly examples 128-135, wherein the sheath is removed from the mandrel.

Example 137: The method of making an expandable sheath according to any example herein, particularly examples 127-136, wherein the heat shrink layer is removed.

Example 138: The method of making an expandable sheath according to any example herein, particularly examples 102-137, further comprising: mounting a proximal end of the sheath to a hub.

Example 139: The method of making an expandable sheath according to any example herein, particularly examples 102-138, wherein an expanded diameter of a central lumen of the inner member is greater in the expanded configuration than an unexpanded diameter of the central lumen of the inner member in the unexpanded configuration, wherein the outer elastomeric member biases the inner member to a return diameter that is substantially identical to the unexpanded diameter.

Example 140: The method of making an expandable sheath according to any example herein, particularly examples 102-138, wherein, when the elongated inner member creases proximate the first and second longitudinal edges into the unexpanded configuration, the second circumferential portion is creased into a folded configuration radially inward of an outer diameter of the first circumferential portion.

Example 141: The method of making an expandable sheath according to any example herein, particularly example 140, wherein the second circumferential portion locally unfolds in response to a radially outward force on the inner surface of the central lumen.

Example 142: The method of making an expandable sheath according to any example herein, particularly examples 140-141, wherein, in the folded configuration, the second circumferential portion includes an overlapping portion and an underlying portion, the overlapping portion position radially outward from the underlapping portion.

Example 143: The method of making an expandable sheath according to any example herein, particularly example 142, wherein, in the folded configuration, an inner diameter of the underlying portion corresponds to an inner diameter of the first circumferential portion.

Example 144: A method of delivering a prosthetic device to a procedure site, the method comprising: positioning an expandable sheath within the vascular system of a subject; introducing a prosthetic device into a lumen of the expandable sheath; advancing the prosthetic device through the lumen of the expandable sheath such that the prosthetic device exerts a radially outward force on an inner surface of an inner member of the expandable sheath and locally unfolds the inner member into an expanded configuration; advancing the prosthetic device further through the lumen and through a distal tip portion of the expandable sheath locally enlarging a lumen of the distal tip portion in response to radial pressure exerted by passage of the prosthetic device; at least partially collapsing the inner member and the distal tip portion after the prosthetic device has passed in response to a radially inward force provided by an outer elastomeric member of the sheath; and delivering the prosthetic device to the procedure site.

Example 145: The method of delivering a prosthetic device to a procedure site according to any example herein, particularly example 144, wherein the prosthetic device is a prosthetic heart valve.

Example 146: The method of delivering a prosthetic device to a procedure site according to any example herein, particularly examples 144-145, wherein the procedure site is an aortic valve.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove and that many modifications and other embodiments are intended to be included within the scope of the appended claims. Moreover, although specific terms are employed herein, as well as in the claims which follow, they are used only in a generic and descriptive sense and not for the purposes of limiting the described invention nor the claims which follow. We, therefore, claim as our invention all that comes within the scope and spirit of these claims.

We claim:

1. An expandable sheath comprising:
an elongated inner member defining a central lumen, a first circumferential portion including a first and second longitudinal edges, and a second circumferential portion extending between the first and second longitudinal edges, the first circumferential portion having a wall thickness greater than a wall thickness of the second circumferential portion;
wherein the elongated inner member is configured to transition from an unexpanded configuration to an expanded configuration in response to a radially outward force on an inner surface of the central lumen;
wherein, in the unexpanded configuration, the elongated inner member is creased proximate the first and second longitudinal edges into a folded configuration such that the second circumferential portion is positioned at least partially radially inward of the first circumferential portion,
wherein an expanded diameter of a central lumen of the elongated inner member in the expanded configuration is greater than an unexpanded diameter of the central lumen of the elongated inner member in the unexpanded configuration.

2. The expandable sheath of claim 1, wherein the elongated inner member includes a plurality of first circumferential portions and a plurality of second circumferential portions extending between the first and second longitudinal edges of the adjacent first circumferential portions.

3. The expandable sheath of claim 1, wherein the elongated inner member includes three first circumferential portions and three second circumferential portions extending between the first and second longitudinal edges of the adjacent first circumferential portions.

4. The expandable sheath of claim 1, wherein, when the elongated inner member creases proximate the first and second longitudinal edges into the unexpanded configuration, the second circumferential portion is creased into a folded configuration radially inward of an outer diameter of the first circumferential portion.

5. The expandable sheath of claim 1, wherein, in the folded configuration, the second circumferential portion includes an overlapping portion and an underlying portion, the overlapping portion positioned radially outward from the underlapping portion,
wherein, when the elongated inner member transitions between the unexpanded configuration and the expanded configuration, the overlapping portion slides along the underlying portion such that an amount of overlap between the overlapping an underlying portions decreases, and increases when the elongated inner member transition between the expanded and unexpanded configuration.

6. The expandable sheath of claim 4, wherein, in the folded configuration, an inner diameter of the underlying portion corresponds to or is less than an inner diameter of the first circumferential portion.

7. The expandable sheath of claim 1, wherein, in the unexpanded configuration, the second circumferential portion includes: a first crease proximate the first longitudinal edge and a second crease proximate the second longitudinal edge; a third crease between the first and second crease; and a fourth crease between the third and second crease.

8. The expandable sheath of claim 1, wherein the second circumferential portion is centered with respect to the wall thickness of the first circumferential portion.

9. The expandable sheath of claim 1, wherein the second circumferential portion is positioned adjacent a top/outer edge of the wall thickness of the first circumferential portion.

10. The expandable sheath of claim 9, wherein, when the elongated inner member is in the expanded configuration, an outer diameter of the first circumferential portion corresponds to an outer diameter of the second circumferential portion.

11. The expandable sheath of claim 1, further including a liner provided within a central lumen of the elongated inner member, wherein the liner is bonded to the elongated inner member, wherein at least a portion of an inner surface of the elongated inner member is bonded to at least a portion the liner by heat processing, laser welding, compression bonding, selective ultrasonic welding, and/or reflow processing.

12. A method of making an expandable sheath, the method comprising:
forming an inner member having portions of varying wall thickness including a first circumferential portion having a first and second longitudinal edges, and a second circumferential portion extending between the first and second longitudinal edges, the first circumferential portion having a wall thickness greater than a wall thickness of the second circumferential portion; and
disposing an outer elastomeric member over the inner member to form the sheath;
wherein the sheath is configured to transition from an unexpanded configuration to an expanded configuration in response to a radially outward force on an inner surface of a central lumen of the inner member,
wherein the outer elastomeric member urges the inner member towards the unexpanded configuration,
wherein, in the unexpanded configuration the elongated inner member is creased proximate the first and second longitudinal edges into a folded configuration such that the second circumferential portion is positioned at least partially radially inward of the first circumferential portion.

13. The method of claim 12, wherein forming an inner member includes forming the second circumferential portion to extend toward a longitudinal axis of the inner member,
wherein forming an inner member includes forming the second circumferential portion to define a semi-circular shape in cross section.

14. The method of claim 12, further including:
disposing a liner within a central lumen of the inner member; and
bonding the liner to an inner surface of the inner member;
wherein at least a portion of an inner surface of the elongated inner member is bonded to at least a portion of an outer surface of the liner by heat processing, laser welding, compression bonding, selective ultrasonic welding, and/or reflow processing.

15. The method of claim 12, further comprising: forming a tapered shape into an elastomeric end portion of the elongate inner member and/or outer elastomeric member
mounting an elastomeric distal tip to a distal end of the sheath by heat processing, laser welding, compression bonding, and/or selective ultrasonic welding, and/or by a chemical and/or mechanical fastener.

16. The method of claim 12, further comprising:
applying a heat shrink layer to an outer surface of the outer elastomeric layer;
mounting sheath on a mandrel, wherein mounting the sheath on a mandrel includes maintaining the second circumferential portion in a configuration having an inwardly projecting curvilinear shape in cross section;
bonding at least one of the inner member, outer elastomeric member, and liner by heat processing at a temperature from about 350° F. to about 550° F. for a time period effective to form a bond between at least a portion of at least one of the inner member, outer elastomeric member, and liner;
removing the sheath from the mandrel; and
removing the heat shrink layer is removed.

17. The method of claim 12, wherein, when the elongated inner member creases proximate the first and second longitudinal edges into the unexpanded configuration, the second circumferential portion is creased into a folded configuration radially inward of an outer diameter of the first circumferential portion.

18. The method of claim 17, wherein, in the folded configuration, the second circumferential portion includes an overlapping portion and an underlying portion, the overlapping portion position radially outward from the underlapping portion.

19. The method of claim 18, wherein, in the folded configuration, an inner diameter of the underlying portion corresponds to an inner diameter of the first circumferential portion.

20. A method of delivering a prosthetic device to a procedure site, the method comprising:
positioning an expandable sheath within the vascular system of a subject;
introducing a prosthetic device into a lumen of the expandable sheath;
advancing the prosthetic device through the lumen of the expandable sheath such that the prosthetic device exerts a radially outward force on an inner surface of an inner member of the expandable sheath and locally unfolds the inner member into an expanded configuration;
advancing the prosthetic device further through the lumen and through a distal tip portion of the expandable sheath locally enlarging a lumen of the distal tip portion in response to radial pressure exerted by passage of the prosthetic device;
at least partially collapsing the inner member and the distal tip portion after the prosthetic device has passed in response to a radially inward force provided by an outer elastomeric member of the sheath; and
delivering the prosthetic device to the procedure site.
